(19) United States
(12) Patent Application Publication
(10) Pub. No.: US 2024/0047042 A1
(43) Pub. Date: Feb. 8, 2024

(54) METHODS AND SYSTEMS FOR GENERATING PERSONALIZED TREATMENTS VIA CAUSAL INFERENCE

(71) Applicant: Evidation Health, Inc., San Mateo, CA (US)

(72) Inventor: Eric Jay DAZA, Menlo Park, CA (US)

(21) Appl. No.: 18/365,899

(22) Filed: Aug. 4, 2023

Related U.S. Application Data
(60) Provisional application No. 63/370,525, filed on Aug. 5, 2022.

(51) Int. Cl. G16H 20/70; G16H 40/67; G16H 20/30; G06N 20/20

(52) U.S. Cl. CPC G16H 20/70; G16H 40/67; G16H 20/30; G06N 20/20

(57) ABSTRACT

Provided herein are systems, methods, computer-readable media, and techniques for generating a personalized recommended intervention for a subject based on causal inference, including: obtaining a first set of time series data and a second set of time series data, the first set of time series data relating to a first variable indicative of a health behavior of the subject, and the second set of time series data relating to a second variable indicative of a health condition of the subject; determining a causal effect of the first variable on the second variable by estimating an average treatment effect, wherein the average treatment effect is estimated by processing the first set of time series data and the second set of time series data using a model-twin randomization method; and generating a personalized treatment or intervention recommendation for the subject to change the health condition based on the causal effect.

### CROSS-REFERENCE

This application claims priority U.S. Provisional Application No. 63/370,525, filed Aug. 5, 2022, which is entirely incorporated herein by reference.

### BACKGROUND

Advances in technology have made it easier to collect large amounts of longitudinal data and determine associations between different sets of data. For example, wearable or ambient devices or sensors may generate volumes of data that are indicative of mental, behavioral, or physical health. If a subject undergoes a treatment, the treatment may affect one or more health outcomes recorded by the wearable or ambient devices or sensors. But beyond acknowledging a statistical association between sets of data collected during treatment, current analysis techniques often do not establish causal links between the sets of data, which would allow for better understanding of a treatment's effects.

### SUMMARY

In one aspect, a method is provided for generating a personalized recommended intervention for a subject based at least in part on causal inference, comprising: (a) obtaining a first set of time series data associated with the subject and a second set of time series data associated with the subject, wherein the first set of time series data relates to a first variable indicative of a health behavior of the subject and wherein the second set of time series data relates to a second variable indicative of a health condition of the subject; (b) determining a causal effect of the first variable on the second variable by estimating an average treatment effect, wherein the average treatment effect is estimated by processing the first set of time series data and the second set of time series data using a model-twin randomization method; and (c) generating a personalized treatment or intervention recommendation for the subject to change the health condition based at least in part on the causal effect determined in (b). In some embodiments, the model-twin randomization method comprises a sequential technique to implement g-formula for estimating the average treatment effect. In some embodiments, implementing the g-formula comprises implementing extensions of the g-formula, wherein the extensions comprise one or both of targeted learning or targeted maximum likelihood estimation. In some embodiments, the sequential technique comprises a simulation-based technique or a Monte Carlo technique. In some embodiments, processing the first set of time series data and the second set of time series data using the model-twin randomization method comprises randomizing the first set of time series data for each time period or time point of the first set of time series data. In some embodiments, the method further comprises running a model-twin through the randomized first set of time series data for a number of iterations until a convergence condition is satisfied. In some embodiments, the model-twin is an outcome model fitted to the first set of time series data and the second set of time series data. In some embodiments, the method further comprises generating, by the model-twin, a predicted value of the second variable at each time point of the second set of time series data. In some embodiments, the method further comprises adding random noise to each predicted value of the second variable. In some embodiments, the method further comprises determining an average period treatment effect (APTE) from at least a subset of each of the predicted values for at least a subset of the set of time points. In some embodiments, the method further comprises estimating a confidence interval for the APTE. In some embodiments, the method further comprise calculating a cumulative average confidence interval, wherein the convergence condition relates to the cumulative average confidence interval. In some embodiments, the model-twin comprises a generalized linear model. In some embodiments, the linear model comprises a generalized linear model. In some embodiments, the model-twin comprises a non-parametric model. In some embodiments, the model-twin comprises a machine learning model. In some embodiments, the machine learning model comprises a random forest. In some embodiments, the first set of time series data is acquired from one or more data-collection instruments that comprise at least one wearable device worn by the subject. In some embodiments, the first set of time series data is indicative of sleep duration and wherein the second set of time series data is indicative of physical activity. In some embodiments, the second set of time series data is indicative of speed of walking. In some embodiments, the second set of time series data is indicative of sleep duration and wherein the first set of time series data is indicative of physical activity. In some embodiments, one or both of the first set of time series data or the second set of time series data is collected daily. In some embodiments, one or both of the first set of time series data or the second set of time series data comprise variables that cause, moderate, or contextualize data comprised in one or both of the first set of time series data or the second set of time series data. In some embodiments, the personalized treatment or intervention recommendation comprises changing health behavior of the subject. In some embodiments, changing the health behavior or the subject comprises estimating a plausible or suggested average treatment effect of the health behavior of the subject on the health condition of the subject.

In another aspect, one or more non-transitory computer-readable media are provided comprising computer-executable instructions that, when executed by at least one processor, cause the at least one processor to: (a) obtain a first set of time series data associated with the subject and a second set of time series data associated with the subject, wherein the first set of time series data relates to a first variable indicative of a health behavior of the subject and wherein the second set of time series data relates to a second variable indicative of a health condition of the subject; (b) determine a causal effect of the first variable on the second variable by estimating an average treatment effect, wherein the average treatment effect is estimated by processing the first set of time series data and the second set of time series data using a model-twin randomization method; and (c) generate a personalized treatment or intervention recommendation for the subject to change the health condition based at least in part on the causal effect determined in (b).

In another aspect, a computer system is provided for generating a personalized recommended intervention for a subject based at least in part on causal inference, comprising: one or more processors; and one or more memories storing computer-executable instructions that, when executed, cause the one or more processors to: (a) obtain a first set of time series data associated with the subject and a second set of time series data associated with the subject, wherein the first set of time series data relates to a first variable indicative of a health behavior of the subject and wherein the second set of time series data relates to a second variable indicative of a health condition of the subject; (b) determine a causal effect of the first variable on the second variable by estimating an average treatment effect, wherein the average treatment effect is estimated by processing the first set of time series data and the second set of time series data using a model-twin randomization method; and (c) generate a personalized treatment or intervention recommendation for the subject to change the health condition based at least in part on the causal effect determined in (b).

Another aspect of the present disclosure provides a non-transitory computer readable medium comprising machine executable code that, upon execution by one or more computer processors, implements any of the methods or techniques above or elsewhere herein.

Another aspect of the present disclosure provides a system comprising one or more computer processors and computer memory coupled thereto. The computer memory comprises machine executable code that, upon execution by the one or more computer processors, implements any of the methods or techniques above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a block diagram of an environment for modeling an outbreak;

FIGS. 2A and 2B show examples of a simulation that of model-twin randomization;

FIGS. 3A and 3B show examples of results of 100 simulations in a simulation study for assessing model-twin randomization;

FIG. 4 shows an example of a flowchart illustrating a method for generating a personalized recommended intervention for a subject based at least in part on causal inference;

FIG. 5 shows an example of a computer system that is programmed or otherwise configured to implement methods provided herein; and

FIG. 6 shows an example of a random forest with a plurality of decision trees.

### DETAILED DESCRIPTION

While various embodiments of the invention have been shown and described herein, it will be obvious to those skilled in the art that such embodiments are provided by way of example only Numerous variations, changes, and substitutions may occur to those skilled in the art without departing from the invention. It should be understood that various alternatives to the embodiments of the invention described herein may be employed.

The ever-increasing abundance of frequently collected, temporally dense single-person small data may allow for extracting personalized insights from this digital individual-level information. However, standard analyses of such intensive longitudinal data merely characterize statistical associations, making conclusions of causal effects generally hard to defend. Causal effects are used to make these insights truly actionable to the extent that one actually expects to see an impact from intervening on a measured factor.

Currently, epidemiologists and econometricians have developed and used causal inference techniques to better inform population-level policies and decisions to improve societal outcomes. However a question remains as to how behavior change scientists can apply these techniques to within-individual observational studies in order to better inform individual-level behavioral interventions and habit-changing practices.

One solution to this question includes conducting within-individual experiments via the mobile phone apps and wearable or implantable sensors that enable small-data collection. These may be simple randomized or forced crossover designs as are commonly used in within-individual studies, that may not use experimental or observational causal inference methods.

In some cases, individuals with a chronic health condition (CHC), which may include common recurring conditions, in particular may benefit from such digitally informed self-experiments as provided by the systems, the methods, the computer-readable media, and the techniques disclosed herein. These CHCs may include migraines, chronic pain, asthma, and irritable bowel syndrome, etc. In some cases, substantial barriers to self-experimentation may exist for these CHCs, highlighting the usefulness of better using dense digital data by extracting insights that are not just “correlational” (e.g., statistically associated) but also plausibly causal in nature.

In some cases, an n-of-1 trial is a randomized single-person crossover trial. For example, one individual undergoes multiple crossover periods with varying treatments. The target population in an n-of-1 trial may be a largely unobserved superset of periods experienced by one person. Specifically, it may be the set of all possible time periods where the subject is at risk for a CHC, and is under at least one of the treatment conditions being studied both during and outside of the n-of-1 trial total study period (e.g., before and after all n-of-1 treatment periods, in the “real world”). Hence, this target population may be referred to as a “population of yourself” or “population-of-1.”

Many biomedical research and clinical trials in particular have successfully employed n-of-1 trials, with trial design, implementation, and analysis guidance available in various texts. Accordingly, some wearable sensors can be used to facilitate n-of-1 trials.

In some cases, single-case experimental designs (SCEDs) in psychology and n-of-1 trials in clinical settings are experimental single-person crossover studies focused on, though not always limited to, one individual. These two types of studies differ in design, but both are used to infer average individual-specific outcomes under different intervention levels. Each average may be taken across a set of consecutive (though, e.g., not necessarily contiguous) time intervals with the same exposure level or treatment level. These intervals may be called phases in SCEDs, and periods in n-of-1 trials.

In some cases, both SCEDs and n-of-1 trials share the same underlying statistical foundations and target estimands. In statistics, summary quantities that describe or apply to repeated measures taken on one study participant may be described as “within-subject” or “within-individual” (examples may include the within-subject sum-of-squares, within-cluster variance, and the intra-cluster or intraclass correlation in mixed-effects or random-effects models and survey sampling.) Hence, collectively, SCEDs, n-of-1 trials, and their observational (e.g., nonexperimental/non-randomized) counterparts may be referred to as “within-individual” studies, similar to “idiographic” studies in psychology.

On the other hand, in some cases, hierarchical or multilevel models may be used in group-level or “nomothetic” studies to infer an average outcome taken across a set of repeatedly measured individuals. Their target population may include a largely unobserved superset of individuals. In the special case of a longitudinal study, longitudinal models may be used to posit and infer a population-average trend taken across a set of individual trends.

In some cases, a single-person crossover observational study may be a non-experimental, non-randomized study of one person, where recurring confounding and selection bias may exist. Disclosed herein is single-person crossover observational study objective to discover plausible causal effects of would-be interventions that may be tested in a subsequent single-person crossover experiment like an n-of-1 trial or SCED.

In some cases, using the Neyman-Rubin-Holland counterfactual framework as a scientific foundation, a single-person crossover observational study analytical framework for inferring a single-person crossover average treatment effect (ATE) may be formulated. This may be within-individual ATE, the average period treatment effect (APTE), based on the n-of-1 trial design that partitions observations into distinct periods, or the single-case experimental design (SCED) design that partitions observations into distinct phases. The APTE may be a recurring average individual treatment effect. The APTE framework may elaborate on the implicit causal assumptions underlying dynamic regression models specified for a single individual. Additionally, this may enable a counterfactual interpretation of repeated effects over n-of-1 trial periods or SCED phases by stating common analytic challenges in terms of potential outcomes, including outcome autocorrelation and time trends (over multiple periods), and intervention carryover effects.

In some cases, the utility of the APTE framework may be appreciated by applying two common causal inference methods, the g-formula (e.g., standardization, back-door or regression adjustment) and propensity-score inverse probability weighting, to infer a possible average effect of an exposure on CHC (e.g., to infer a possible average effect of physical activity on weight). Some techniques towards causal prediction of electricity consumption may use a prognostic-score technique and develop the large-sample theory needed to conduct inference on the APTE if exposure effects do not carry over from one period to the next.

In some cases, the systems, the methods, the computer-readable media, and the techniques of the present disclosure may utilize machine learning techniques to compute ATEs for users in different population cohorts. In some cases, the users of a cohort may be similar (e.g., sharing similar demographics), and users within a cohort may be exposed to different health behaviors, health conditions, or interventions. The machine learning techniques trained model may be used to estimate, e.g., effectiveness of different treatments to perform comparative analyses across treatments/preventative care measures (which can later be prospectively validated). In some cases, the machine learning techniques use non-parametric techniques. In some cases, causal inference techniques such as matching, propensity-score matching or weighting, the g-formula, or augmented inverse probability weighted estimator (a combination of the g-formula and propensity-score weighting) can be used to estimate ATEs for each cohort. In some cases, the machine learning techniques may gather propensity estimates from, e.g., health organizations.

In some cases, the systems, the methods, the computer-readable media, and the techniques disclosed herein may be applied to various fields including, for example, latent health states, acute illness, recovery trajectories, CHCs, intervention assistance, fitness tracking, functional data analysis, digital phenotyping, personal sensing, etc.

In some cases, the systems, the methods, the computer-readable media, and the techniques disclosed herein may be applied to fields outside of health. In such cases, a user may include an athlete, audience member, shopper, body part, animal, vehicle, sports team, organization, political party, institution, country, geographic region, financial instrument, recurrent group behavior, geologic phenomenon, etc. In some cases, rather than a user specifically, the systems, the methods, the computer-readable media, and the techniques disclosed herein may more broadly apply to any observational unit (e.g., an object, a subject, a phenomenon, etc.) that goes through various experiences (e.g., recurring experiences, related experiences, etc.) overtime. As such, fields of use may include, finance, government, politics, ecology, geology, climate, entertainment, athletics, astronomy, economics, manufacturing, etc.

### Certain Notations and Definitions

As used in this specification and the appended claims, various notations are used and will now be discussed. Random variables and fixed values are written in upper-case and lower-case, respectively. Let p(A=a) denote the probability mass or density of random variable A at a, with shorthand p(a). For any random variable B, let B|A denote the relationship “B conditional on A,” with shorthand B|a for B|A=a. Let B [symbol] A denote statistical independence of B and A. Let E (⋅) denote the expectation operator, and let I(⋅) denote the identity function such that I(b)=1 if expression b is true and I(b)=0 otherwise. Turning next to set notation, let {a} denote a set with elements {a₁, a₂, . . . }. Likewise, let a denote a vector with elements denoted with commas as in (a₁, a₂, . . . ), or without commas as in (a₁a₂ . . . ). Assume vectors multiplied together are conformable; e.g., a 1×p random vector A and its p×1 coefficient vector β_{A} can be vector-multiplied as A β_{A}. Let ({Aₜ})=(A1, A2, . . . ) denote a stochastic process or time series (e.g., vector of ordered random variables or vectors). For any non-empty set S, Let {a} denote the set of all permutations of all possible values of the vector a, not including the empty set Ø.

Unless otherwise defined, all technical terms used herein have the same meaning as commonly understood by one of ordinary skill in the art to which the present subject matter belongs.

As used in this specification and the appended claims, the terms “artificial intelligence,” “artificial intelligence techniques,” “artificial intelligence operation,” and “artificial intelligence algorithm” generally refer to any system or computational procedure that may take one or more actions to enhance or maximize a chance of achieving a goal. An example of such a goal is to mathematically or computationally model the probabilistic relationship between an exposure and an outcome like a CHC. The term “artificial intelligence” may include “generative modeling,” “machine learning” (ML), or “reinforcement learning” (RL).

As used in this specification and the appended claims, the terms “machine learning,” “machine learning techniques,” “machine learning operation,” and “machine learning model” generally refer to any system or analytical or statistical procedure that may progressively improve computer performance of a task. An example of such a task is to mathematically or computationally model the probabilistic relationship between an exposure and an outcome like a CHC.

As used in this specification and the appended claims, “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

As used in this specification and the appended claims, when the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

As used in this specification and the appended claims, when the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used in this specification, “or” is intended to mean an “inclusive or” or what is also known as a “logical OR,” wherein when used as a logic statement, the expression “A or B” is true if either A or B is true, or if both A and B are true, and when used as a list of elements, the expression “A, B or C” is intended to include all combinations of the elements recited in the expression, for example, any of the elements selected from the group consisting of A, B, C, (A, B), (A, C), (B, C), and (A, B, C); and so on if additional elements are listed. As such, any reference to “or” herein is intended to encompass “and/or” unless otherwise stated.

As used in this specification and the appended claims, the indefinite articles “a” or “an,” and the corresponding associated definite articles “the” or “said,” are each intended to mean one or more unless otherwise stated, implied, or physically impossible. Yet further, it should be understood that the expressions “at least one of A and B, etc.,” “at least one of A or B, etc.,” “selected from A and B, etc.” and “selected from A or B, etc.” are each intended to mean either any recited element individually or any combination of two or more elements, for example, any of the elements from the group consisting of “A,” “B,” and “A AND B together,” etc.

As used in this specification and the appended claims “about” or “approximately” may mean within an acceptable error range for the value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where values are described in the application and claims, unless otherwise stated the term “about” meaning within an acceptable error range for the particular value may be assumed.

### Examples of Use Cases

In some cases, the systems, the methods, the computer-readable media, and the techniques disclosed herein may generally implement model-twin randomization to estimate within-individual average treatment effect, a causal effect estimation based on “single-person small data.”

In some cases, temporally dense single-person “small data” are becoming more accessible with adoption of mobile apps and wearable sensors. Many caregivers and self-trackers may leverage these data to help a specific person change their behavior to achieve desired health outcomes. This may include discerning possible causes from correlations using a person's own observational time series data. The systems, the methods, the computer-readable media, and the techniques disclosed herein estimate within-individual average treatment effects of physical activity on sleep duration, and vice-versa. Disclosed herein is a model-twin randomization (MoTR; “motor”) for analyzing a person's intensive longitudinal data. The MoTR may be an application of the g-formula (e.g., standardization, back-door adjustment) under serial interference. The MoTR may estimate stable recurring effects, as is done in n-of-1 trials and single case experimental designs. As demonstrated herein, the systems, the methods, the computer-readable media, and the techniques disclosed herein improve upon other correlation-based techniques (e.g., that characterize statistical associations, not causal effects) by using causal inference to make better personalized recommendations for health behavior change.

In some cases, the systems, the methods, the computer-readable media, and the techniques disclosed herein provide for inferring causal relationships between time series sets of intensive longitudinal health data; for example, app-based survey data (e.g., patient-reported outcomes and other clinical outcome assessments) and wearable or ambient device or sensor data. Wearable or ambient device or sensor data may include physical or biometric statistics (e.g., a statistic is defined as a single measurement in a sample, or a calculated summary or aggregate taken over a set of single measurements in a sample) such as length of time sleeping, heart rate, and step count. These statistics may be indicative of a subject's physical health For example, the statistics may be indicative of recovery from an acute event, such as an illness or injury. The statistics may be indicative of the acute event itself. For example, a user's step count may decrease during an illness.

In some cases, the systems, the methods, the computer-readable media, and the techniques disclosed herein provide for generating a “model twin” of a subject to infer the causal relationship of an observed, non-randomized, non-forced, or natural exposure on a recurring health outcome (e.g., from a CHC). The model twin is a statistical model of a relationship between a first set of time series values and a second set of time series values.

After the model twin is generated (e.g., by fitting the model), the disclosed technique may produce an average period treatment effect (APTE) estimate describing an effect produced by the first set of time series values on the second set of time series values. This APTE may be produced by randomizing the first set of time series data, using the model twin to predict values of the second set of time series data from the randomized first set of data. The calculation of the APTE may be repeated until a convergence condition is reached.

Temporal considerations such as autocorrelation, time trends, carryover, and exogenous factors may complicate analysis by creating serial interference over time. The model-twin randomization technique as used herein may mitigate the effects of this temporal interference.

In some cases, the causal inference techniques disclosed herein may be used to determine the impact of an adaptive treatment on recovery from an acute illness or health condition. A treatment may be associated with a time series set of app-based survey data or wearable or ambient device or sensor data. For example, a person who has suffered an injury may be prescribed a regimen of daily exercises which may comprise body motions recorded by a wearable device. A person's recovery may be tracked by monitoring the person's step count, as increasing a number of steps performed may be associated with recovery. The model-twin randomization technique described herein may be used to estimate a plausible effect of daily performance of the exercise on a change in average step count, to determine a potential causal relationship between the two. This suggested effect may subsequently be used to adjust the exercise regimen to achieve a better average step count.

In some cases, the causal inference techniques disclosed herein may be used to determine causal relationships between behavioral changes and changes in physical health. For example, the system may collect time series data related to sleep quality and step count. The disclosed model-twin randomization technique may determine a causal relationship between the time series data relating to sleep quality and the time series data related to step count.

The systems, the methods, the computer-readable media, and the techniques disclosed herein may present a Monte Carlo technique called model-twin randomization (MoTR) for estimating the APTE. This includes modeling the outcome as a function of all causes, and then running this “model-twin” through a simulated n-of-1 trial by randomizing the exposure at each time point to generate an effect estimate.

The systems, the methods, the computer-readable media, and the techniques disclosed herein may employ causal inference concepts such as (1) an autoregressive carryover modeling technique for estimating or predicting a time series of potential outcomes that is stable in the long run, and (2) MoTR as a Monte Carlo (numerical) technique for estimating an APTE from these stable time series that itself is stable in the long run. Performance of these techniques will be demonstrated herein. Further, applications of these techniques, e.g., use of MoTR (and a complementary propensity-score technique) to estimate the APTE of sleep duration on steps per minute are disclosed herein.

### Examples of Environments

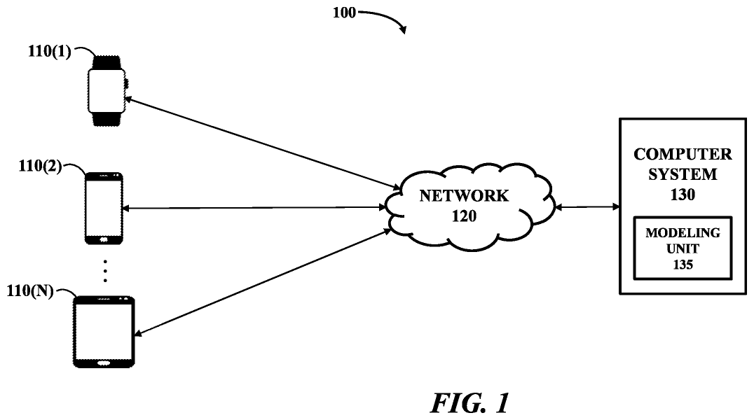

FIG. 1 is a block diagram of an environment 100 for generating a personalized recommended intervention for a subject based at least in part on causal inference, in accordance with some cases. The environment 100 of FIG. 1 includes one or more data-collection instruments 110(1)-110(N), which in some cases may be data-collection instruments as in this example, a network 120, and a computer system 130 with a modeling unit 135. In some cases, the data-collection instrument may be non-electronic recording equipment with data that are then entered into an electronic device. For example, non-electronic recording equipment can be a pen and a writing pad, a chalkboard, or a glass board. At a high level, the data-collection instruments 110(1)-110(N) may be used by one or more users (e.g., humans, animals, etc.) to collect or provide, via the network 120, data about the one or more users to the computer system 130 so that the computer system 130, using the modeling unit 135, can predict risk of an acute illness to the one or more subjects or a population (e.g., including at least a portion of the one or more subjects).

The data-collection instruments 110(1)-110(N) may be one or more electronic devices. In some cases, the electronic devices may be desktop computers. In some cases, the electronic devices may be mobile devices. For example, the mobile devices may be smartphones (e.g., the data-collection instrument 110(2)) or tablets (e.g., the data-collection instrument 110(N)). In some cases, the electronic devices may be wearable or ambient devices or sensors. For example, the wearable or ambient devices or sensors may be smartwatches (e.g., the data-collection instrument 110(1)), smartbands (e.g., a Fitbit® device), smartclothing, smart jewelry, smartshoes, environmental sensors, or the like. In some cases, the electronic devices may be a laptop or desktop computer. In some cases, the electronic devices may be gaming devices (e.g., the gaming device may collect data as part of a game or may reward users based on certain collected data or outcomes). In some cases, the data-collection instruments 110(1)-110(N) may include an intraday sensor that may be used to characterize average daily trends across periods (e.g., analogous to longitudinal trends or trends in repeated measures across individuals). In general, the data-collection instruments 110(1)-110(N) may be any device suitable for collecting data such as wearable or ambient device or sensor data, responses to queries, geographical or meteorological data (e.g, location, environmental conditions, etc.), demographical data (e.g., age, height, weight, ethnicity, gender, etc.), medical data (e.g., blood pressure, glucose levels, body temperature, etc.), short/long-term trends, summaries, and statistics (e.g., averages, medians, maximums, minimums, ranges, etc.), or any other health data or the like. For example, the data-collection instruments 110(1)-110(N) may be any one or more of desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, vehicles, televisions, exercise equipment, video players, digital music players, booklet tablet computers, slate tablet computers, convertible tablet computers, or the like. Each of the data-collection instruments 110(1)-110(N) may be linked to, and collect data from, a single user. Each the data-collection instruments 110(1)-110(N) may be linked to, and collect data from, multiple users (e.g., a household).

As described, the data-collection instruments 110(1)-110(N) are data-collection instruments that can be wearable or ambient devices or sensors or other device capable of providing physical (e.g., medical, activity, nutrition, sleep, etc.) statistics about one or more users. For example, the data-collection instruments 110(1)-110(N) can be a dedicated fitness tracker, a pedometer, a wrist-worn sleep tracker, a sleep-tracking mattress pad, a smart scale, a blood pressure monitor, an ambient air particle sensor, a CPAP machine, a smart watch, smartphone, or mobile device (e.g., a tablet computer or a personal digital assistant (PDA)) with physical statistic monitoring functionality. For example, the data-collection instruments 110(1)-110(N) can be a smartphone of one or more users with an installed physical statistic monitoring application using one or more sensors of the smartphone to measure steps, activity, movement, sleep time, or other physical statistics. In some cases, a user may be associated with multiple devices of the data-collection instruments 110(1)-110(N) measuring overlapping or distinct physical statistics about the user. For example, a smartphone may measure sleep behavior or sleep efficiency of the user (e.g., based on when the user uses the device), a pedometer may measure step counts of the user, and a smart exercise machine may measure blood pressure of the user. The health behavior data gathered by the data-collection instruments 110(1)-110(N) can be sent to the computer system 130 directly from the data-collection instruments 110(1)-110(N), manually uploaded to the computer system 130 by the associated users or transmitted via a third-party system to the computer system 130. For example, a user may authorize a third-party service associated with the data-collection instruments 110(1)-110(N) to transmit physical activity data to the computer system 130. In some cases, a user can interact with the computer system 130 via the data-collection instruments 110(1)-110(N). For example, a user may be able to update information about themself (e.g., physical statistics, health conditions, behavior, etc.) stored in the computer system 130 via the data-collection instruments 110(1)-110(N). In some cases, a user can interact with the data-collection instruments 110(1)-110(N) via an external device (not shown; e.g., a laptop, a smartphone, etc.). For example, the user may configure settings the data-collection instruments 110(1)-110(N) through the external device (e.g., turn one or more of the data-collection instruments 110(1)-110(N) on/off, change a sampling rate, etc.). In some cases, a user may further be able to provide feedback relating to one or more estimates or predictions generated using the computer system 130 or manually report health information to the computer system 130 via the data-collection instruments 110(1)-110(N). For example, In some cases, the user may, e.g., through one or more of the data-collection instruments 110(1)-110(N), report to the computer system 130 that they have received a treatment or intervention, have a certain health condition, etc.

Each user of the data-collection instruments 110(1)-110(N) may be a member of a population monitored by the computer system 130. In some cases, each user is associated with one or more of the data-collection instruments 110(1)-110(N) measuring physical statistics of that user. For example, the data-collection instruments 110(1)-110(N) can measure a user's resting heart rate (RHR) over time, a daily number of steps (or other measure of activity level such as distance walked), and sleep statistics (such as duration of sleep, number of times sleep was interrupted, sleep start and end times, etc.) for the user. Recorded physical statistics (e.g., walking data, sleeping data, exercise data, nutrition data, etc.) from the data-collection instruments 110(1)-110(N) may be time series data and may be stored as health behavior data and sent by the data-collection instruments 110(1)-110(N) to the computer system 130, or analyzed on the data-collection instrument itself. For example, health data may be analyzed for correlations, statistical associations, and causation on each of the data-collection instruments 110(1)-110(N), with or without being sent to the computer system 130. For example, the data-collection instruments 110(1)-110(N), using their own modeling unit, may generate personalized treatment or intervention recommendations for a user within a population based on health behavior received from that user. In some cases, the modeling unit of the health data-collection instruments 110(1)-110(N) may predict a health condition for the user based at least in part on the health behavior for the user. In some cases, predicting the health condition for the user may inform the generation of the personalized treatment or intervention recommendations for the user. In some cases, the modeling unit of the health data-collection instruments 110(1)-110(N) may implement a model-twin randomization method. In some cases, the model-twin randomization technique comprises a sequential technique to implement g-formula for estimating average treatment effect. In some cases, the sequential technique comprises a simulation-based technique or a Monte Carlo technique. In some cases, some or all health behavior data is collected as time series data, or periodically recorded measurements of physical statistics of the user over time The frequency of measurements included in the physical statistics data sent to the computer system 130 can depend on the data-collection instruments 110(1)-110(N), user preference selections, or the type of health behavior data being collected. For example, the data-collection instruments 110(1)-110(N) may send time series data for average RHR multiple times per day, but send hours slept data once per day. In some cases, the data-collection instruments 110(1)-110(N) may send health behavior data to the computer system 130 frequently, for example, minutely, hourly or in real time.

The data-collection instruments 110(1)-110(N) (and, in some cases, users of the data-collection instruments 110(1)-110(N)) may communicate with the computer system 130 over the network 120. The network 120 may be a network or system of networks connecting the computer system 130 to the data-collection instruments 110(1)-110(N). The network 120 may comprise any combination of local area or wide area networks, using wired or wireless communication systems. In some cases, the network 120 uses standard communications technologies or protocols. For example, the network 120 can include communication links using technologies such as Ethernet, 3G, 4G, CDMA, WIFI, and Bluetooth. Data or information exchanged over the network 120 may be represented using any suitable format, such as hypertext markup language (HTML) or extensible markup language (XML). In some cases, all or some of the communication links of the network 120 may be encrypted using any suitable technique or techniques. In some implementations, the network 120 also facilitates communication between the computer system 130, the data-collection instruments 110(1)-110(N), and other entities of the environment 100 such as the modeling unit 135, users (not shown) or other external devices (not shown).

The computer system 130 may comprise computer devices such as a server, server cluster, distributed server, or cloud-based server capable of predicting health conditions, statistics, risks, etc. For example, the computer system 130, using the modeling unit 135 (which may be the same as or similar to the modeling unit of the health data-collection instruments 110(1)-110(N)), may generate personalized treatment or intervention recommendations for a user within a population based on health behavior received from that user. In some cases, the modeling unit 135 may predict a health condition for the user based at least in part on the health behavior for the user. In some cases, predicting the health condition for the user may inform the generation of the personalized treatment or intervention recommendations for the user. In some cases, the modeling unit 135 may implement a model-twin randomization method. In some cases, the model-twin randomization technique comprises a sequential technique to implement g-formula for estimating average treatment effect. In some cases, the sequential technique comprises a simulation-based technique or a Monte Carlo technique.

In some cases, the computer system 130 gathers health behavior data about a set of users within a population (e.g., through data from one or more of the data-collection instruments 110(1)-110(N)). Health behavior may include physical statistics that may be measurements characterizing a user's activity level or current health state (such as from the data-collection instruments 110(1)-110(N), or other sources) For example, physical statistics can include measurements of the user's vital signs such as body temperature, resting heart rate (RHR), blood pressure, current heart rate (for example, presented as a time series), heart rate variability, respiration rate, or galvanic skin response, measurements of user activity such as daily number of steps, distance walked, time active, or exercise amount, sleep statistics such as time slept, number of times sleep was interrupted, or sleep start and end times, or other similar metrics.

The computer system 130 can analyze received health behavior data to extract learned features or generate a learned representation of the health behavior data using the modeling unit 135. In some cases, the learned representation generated by the modeling unit 135 may store a transformed, modified, or compressed version of raw health behavior data. This version of the raw health behavior data (or wearable or ambient device or sensor data) may preserve richness of information and useful features that may be used to identify trends or outliers among data gathered across a large population, predict health conditions, segment, cluster, or categorize data from different users, or the like.

In some cases, outputs (e.g., predictions of health conditions, recommendations of personalized treatment or interventions, etc.) from the computer system 130 may be shared. For example, the outputs from the computer system 130 may be shared with the corresponding user. In another example, the outputs from the computer system 130 may be shared with researchers or a laboratory (e g., for studying health conditions), with consent from the corresponding user. In another example, the outputs from the computer system 130 may be shared with healthcare providers (e.g., the corresponding user's primary care physician), with consent from the corresponding user. In cases where the outputs from the computer system 130 are shared with the healthcare providers, the healthcare providers may maintain a computing system such as a server, set of servers, server cluster, etc. which can create or modify an individual treatment plan or perform interventions based on predicted health conditions generated using the computer system 130. For example, the computing system of the healthcare providers can be managed by a medical provider, doctor, or other entity providing medical care to a user for a health condition.

### Examples of Sleep Studies

The systems, the methods, the computer-readable media, and the techniques disclosed herein may be applied to sleep studying, in some cases. The timing of sleep and wake is believed to be regulated by 2 main underlying processes: the homeostatic drive and the circadian rhythm. The homeostatic drive is a use-state negative feedback process that accumulates over periods of wakefulness and dissipates over periods of sleep The circadian rhythm is an intrinsic set of biological oscillations with approximately 24-hour (e.g., “circa” means about; “diem” means day) periodicity. Each of these biological processes may be regulated by not only the primary sleep period, but also by an organism's behaviors throughout the waking period. From the standpoint of the sleep homeostasis, the primary somnogen, adenosine, is a breakdown product of the energy molecule adenosine triphosphate (ATP), indicating that it is not just the amount of preceding wakefulness, but the intensity of that wakefulness (e.g., highly active vs. sedentary) that dictates the subsequent sleep drive. Similarly, there are a number of environmental exposures and biological processes (cumulatively termed zeitgebers or “time givers”) that help synchronize the body's intrinsic biorhythms to meet the organismal needs. Most of these signals from dietary intake to light level and color variation are integrated via a “master clock” in the hypothalamus to orchestrate the molecular clocks present in every other cell of the body.

The aforementioned physiologic processes may, in some cases, help to regulate the intrinsic sleep-wake state neurocircuitry in order to meet the needs of the organism (e.g., keeping nocturnal animals up at night). However, there may be a wide range of variation in how these processes play out, even within species. Genome-wide association studies (GWAS) in humans have discovered polygenic contributions to the wide range of variation in typical sleep duration/need and intrinsic circadian phase (e.g., “chronotype”). However, it seems that the intrinsic organismal needs also reflect the function that sleep serves in maintaining health. For example, families of individuals with mutations conferring short sleep need seem to sleep more effectively than others, as is evidenced by their physiologic and functional resilience to very short sleep durations. Studies of transgenic mice showing less neurodegeneration in response to chronic sleep restriction provide further evidence. Taken together, there seems to be not only a genetically determined setpoint for sleep need and timing, but also an individual-specific resilience to perturbations of the sleep-wake system. The current gold-standards of sleep physiologic measurements are quite impractical: from the resource-intensive and disruptive nature of sleeping in a research laboratory to gather neurophysiologic, cardiopulmonary, and behavioral signals, to the highly controlled environment and inconvenient sampling requirements needed to isolate true circadian biomarker measurements (e.g., dim-light melatonin onset). On the other end of the spectrum, affording more real-world convenience at scale, self-report sleep and circadian measures such as the Morningness-Eveningness questionnaire, Munich Chronotype Questionnaire, and the Consensus Sleep Diary are plagued with recall bias and inaccuracy.

Toward this end, the systems, the methods, the computer-readable media, and the techniques disclosed herein that are able to gather sufficiently accurate data offer a unique opportunity for exploring the interrelationship between sleep and wake. The systems, the methods, the computer-readable media, and the techniques disclosed herein may overcome the challenge that what is typically gained in convenience comes at the expense of what is lost in fidelity. For example, sleep-tracking technologies that depend on non-electroencephalographic signals may not accurately recapitulate in-lab polysomnograms (although such sensors often perform reasonably well in their general estimation of the daily amounts of sleep and wake). Similarly, different activity trackers have varying accuracy, depending upon context. However, despite the known limitations in accuracy in how wearables measure both sleep and wake activities, their consistency in measurement longitudinally is core to the potential value they bring to understanding the impact of deliberate interventions or unintended changes within an individual. For these reasons, examining sleep and how it relates to other behavioral metrics by applying the systems, the methods, the computer-readable media, and the techniques disclosed herein to data measured by wearable sensors is a strong target application for idiographic causal inference methods. For example, with the systems, the methods, the computer-readable media, and the techniques disclosed herein, one can examine varying the exposure to factors that impact the physiologic sleep drive to infer the APTE of that factor on sleep.

### Examples of Causal Inference

In some cases, a data-generating function (DGF) or mechanism (or simply mechanism) may be a true, unknown equation that relates input variables or predictors to an outcome variable. In some cases, a model may be a statistical equation that is fit to data, which may or may not approximate the DGF. This distinction reflects the famous Box aphorism that “all models are wrong, but some are useful.”.

Turning to causal inference, let Y represent a variable occurring after a binary variable X with support s∈{0, 1}. If E(Y|X=0)≠E(Y|X=1) when X is randomized, then there may be a direct effect of X on Y, and X may be a direct cause of Y. Let W represent the set of all other direct or indirect causes of Y that may also be direct or indirect causes of X, or may contextualize (e.g., modify or moderate) the effect of X on Y.

In some cases, an indirect cause affects the outcome through other mediating factors, forming a causal path from the in-direct cause to the outcome through the mediators. Causes of both X and Y confound causal interpretations of the statistical dependence between X and Y, and hence may be called confounders. An effect contextualizer may not be directly manipulable, but the effect of X on Y may vary across the contexts it represents (e.g., day of week).

The observable outcome Y may then be written as a function of X, W, and completely random error ε (e.g., due to random between-measurement variation). Let Y=g(X, W,ε) represent this structural causal mechanism (e.g., true “structural causal model” that generates the data); for example, g(X, W, ε)=β₀+β₁X+Wβ₂+ε.

Now let Y^{s} denote the value of Y corresponding to s. This is the outcome that may be observed whenever X=s, and hence may be called the potential outcome (PO) of an exposure variable X. For x∈{0, 1}, the relevant potential outcomes may be Y¹ and Y⁰. The potential outcome Y^{s} can then be written as a function of W and ε. Let Y^{s}=gₛ(W, ε) represent this PO mechanism. In design-based or randomization-based causal inference, the randomness in Y may be assumed to result only from the assignment of X to 1 or 0; hence, the POs may be treated as fixed values (e.g., implying W is also treated as fixed), and so may be written simply as y¹ and y⁰.

The observed outcome Y, intervention of interest X, and potential outcomes {Y^{s}} may be related by the equivalence Y=Σ₍ₛ₎Y^{s}I(X=s), with shorthand Y=Y^{X}. This equivalence may be called causal consistency, and may simply state that the observed outcome is equal to its corresponding PO (e.g., statistical consistency, the asymptotic unbiasedness of an estimator, may simply be referred to as “consistency”). Recalling the earlier example, because x∈{0, 1}, then Y=Y¹X+Y⁰(1−X). Under causal consistency, one natural set of PO mechanisms may be Y¹=β₀+β₁+Wβ₂ε and Y⁰=β₀+Wβ₂+ε. The randomization-based counterpart to this model-based or superpopulation-based technique involves the same equations, but with fixed w and ε.

Causal consistency formalizes the fundamental problem of causal inference that the observable PO may be the one corresponding to X=s. Hence, the term counterfactual outcome (or simply counterfactual) is often used to refer to Y^{x}′ for x′≠x. Accordingly, “counterfactuals” and “potential outcomes” may be used synonymously.

Statistical causal inference may involves four conditions. When the POs occur independently of X, the assignment mechanism may be ignorable, and such ignorability or unconfoundedness holds. We will see that this condition is implied under randomization. For example, {Y^{s}} [symbol] X if X is randomized. If the POs occur independently of X given all other causes W, then conditional ignorability may hold. For example, {Y^{s}} [symbol] X|W.

Another condition, which may be referred to as positivity or overlap, is the empirical requirement that all elements in the support of X co-occur with all other causes. For example, if w∈{0, 1}, then positivity holds if there is at least one observation for each unique pair of w and x. That is, positivity holds if each element of {(w, x)}={(0, 0), (0, 1), (1, 0), (1, 1)} is observed in the data. Positivity may be required for estimating the average treatment effect.

Another condition may be the stable unit treatment value assumption (SUTVA). A key component of this condition is that the POs of a given individual are not affected by any other individual's treatment assignment. That is, there is no interference between individuals. Formally, let {Y_{i′}¹,Y_{i′}⁰} denote the PO sets of distinct individuals i and i′, respectively.

In some cases, if interference exists, then individual i instead has POs {Yᵢ¹¹, Yᵢ¹⁰, Iᵢ⁰¹, Yᵢ⁰⁰}, where Yᵢ^{s}^{ᵢ}^{s}^{_{i′}} denotes the potential outcome if individual i receives treatment sᵢ, while individual i′ receives treatment s_{i′}. Likewise, this holds for individual i′. Causal consistency in this case is defined as

$$Y = {\sum }_{\{ {s}_{i} , {s}_{{i}^{′}} \cdot \}} {Y}^{{s}_{i} {s}_{{i}^{′}}} I ( {X}_{i} = {s}_{i} , {X}_{{i}^{′}} = {s}_{{i}^{′}} ) ,$$

with shorthand Y=Y^{X}^{ᵢ}^{X}^{_{i′}}.These studies may use careful characterization, selection, and estimation of the relevant estimand. In some cases, autocorrelation and treatment carryover from past periods may generate serial interference in single-person crossover studies; e.g., “temporal interference.”

Another condition may aid in establishing effect transportability (e.g., generalization of a treatment effect to a non-experimental setting) and may be referred to as distributional stability or invariance. In some cases, invariance holds if the distribution of the outcome conditional on some subset of predictors does not change across all possible environments or intervention regimes (e.g., randomized vs. observed), which may be referred to as randomization invariance in the special case wherein randomization status neither affects the outcome nor is affected by any confounders.

In some cases, if randomization invariance holds, then randomization status may be an instrumental variable that satisfies the exclusion restriction. That is, randomization status may be associated with the exposure, but not with any confounders, the outcome, or variation in the outcome not explained by the exposure or confounders. This property of randomization may be exploited to estimate single-person crossover personalized average causal effects.

### Examples of Potential Outcomes

In some cases, the PO framework is generally not needed to conduct causal inference in simple or straightforward cases. For example, it may be possible to simply evaluate the model Y=β₀+β₁X+Wβ₂+ε directly when X is randomized. However, the power of the PO framework, may be in handling the complexity of commonplace cases that have become particularly ubiquitous in the era of data science. These span both observational studies with their non-randomized predictors (e.g., convenience samples, real-world data, health claims, medical records, user behavior) and experimental studies with their randomized interventions (e.g., A/B testing, experimentation, network effects, etc.).

Thinking in terms of POs also implicates techniques of statistical inference that are far from obvious when concerned with how to statistically predict or model an outcome—a primary focus of modern data science and machine learning. For example, the PO framework allows causal inference to be understood as a missing data or survey sampling problem, or to be conducted via randomization-based inference (in contrast to the superpopulation-based inference central to statistical modeling and machine learning). This key insight is a reason for propensity score matching and inverse probability weighting (IPW) techniques. POs also encourage development, implementation, and detailed characterization and analysis of discrete interventions that are intuitive and actionable (e.g., “do A=1 or A=0”).

### Examples of Average Treatment Effect

In some cases, in a nomothetic study, participant i∈{1, . . . , n} with Wᵢ=w and εᵢ=ε may be considered to have a set of fixed POs {yᵢ^{s}} with the same number of elements as {s}, as in design-based or randomization-based causal inference. That is, individual i has the two fixed POs yᵢ¹ and yᵢ⁰, and a difference between POs yᵢ¹ and yᵢ⁰ that may be referred to as an individual treatment effect (ITE).

The superpopulation-based counterparts to this randomization-based definition may involve the same quantities, but with random W and ε, yielding random POs Yᵢ¹ and Yᵢ⁰. Here, the randomness in Y may additionally be assumed to result from other mechanisms such as random sampling of study participants, or random variation over time (e.g., even just throughout the study period). In some cases, time-series-based setting uses the assumption that at least some confounders are realizations of randomly varying quantities.

While of primary interest in some cases, the ITE may not be identifiable due to the fundamental problem of causal inference. However, if X is randomized for all individuals, then E(Y^{s}) (e.g., the mean PO taken across all individuals) may be identifiable using only observed data because, by causal consistency for a randomized X,

$$\begin{matrix}E ( Y \vert X = x ) = E ( {Y}^{X} \vert X = x ) \\ = E ( {Y}^{x} \vert X = x ) \\ = E ( {Y}^{x} )\end{matrix} . \qquad ( Equation 1 )$$

A difference between E(Y₁) and E(Y⁰) is called an average treatment effect (ATE). Here, the expectations are taken over all study participants. In randomization-based causal inference, E(Y^{s}) is taken across all study participants with unchanging confounders, contextualizers, and errors. In the superpopulation-based framework, E(Y^{s}) is taken across the superpopulation of all possible realizations of each study participant (e.g., the supports of Wᵢ and εᵢ) for all participants.

Recall Yᵢ¹=β₀+β₁+Wᵢβ₂+εᵢ and Yᵢ⁰=β₀+Wᵢβ₂+εᵢ. First, note that the ITE specified as Yᵢ¹−Yᵢ⁰ is equal to β₁, regardless of individual. This is an important and common assumption in nomothetic causal inference when there is no interference: the ITEs are all equal to the same constant quantity, even though each potential outcome may be random. In general, assume that the ITE is a constant for each individual, as it is here. Indicating that this is a fixed quantity that is nonetheless comprised of random components may use the notation ofδᵢ^{ITE}=Yᵢ¹−Yᵢ⁰.

In this example, the ATE specified as δ^{ATE}=E(δ^{ITE})=E(Y¹−Y⁰)=E(Y¹)−E(Y⁰) is also constant and equal to β₁. This expectation is taken over all individuals, who all have δᵢ^{ITE}=β₁, when X is randomized for all individuals. Hence, the ITEs may all be equal to the ATE.

In some cases, an ATE that varies based on some elements of W is called a conditional ATE (CATE) for heterogeneous treatment effects; e.g., the ATE is heterogenous across subgroups defined by values or levels of W. The CATE is a more realistic estimand in many cases, and an idiographic type of CATE may be estimated using real data.

The ATE may be a primary estimand of interest in a randomized study like a randomized controlled trial (RCT) or A/B tests because all other causes W need not be observed in order to estimate the difference between E(Y¹) and E(Y⁰). That is, if X is randomized for (e.g., randomly assigned to) each individual, then estimates of E(Y|X=s), instead of E(Y^{s}), can be used to estimate the ATE without needing to observe W or know how it functionally relates to Y. This result conveys the power of randomization as a tool for elucidating causal mechanisms, provided the assumption that the ITEs are all identical holds true.

In an observational study, it does not generally follow that E(Y|X=x)=E(Y^{X}). This may be because W may also affect X. When X is not randomized, Equation 1 may hold true up to E(Y|X=x)=E(Y^{X}|X=x). Hence, estimates of E(Y|X=1) and E(Y|X=0) cannot be used to estimate the ATE. The effect of W on X may confound straightforward estimation of the ATE, so W may be called a confounder. If all confounders are observed, standard techniques to estimating the ATE from observational data can be used.

### Examples of Average Period Treatment Effects

In some cases, in an idiographic study, a single participant i may be measured repeatedly over phases or periods t∈{1, . . . , m}. For now, the i index will be dropped as the following may address one participant.

Let Yₜ represent a recurring variable occurring after a recurring binary variable Xₜ. Let Wₜ represent the set of all other causes of Y_{y} that may also be causes of Xₜ. The individual at period t with Wₜ=w and εₜ=ε may have a set of fixed POs yₜ¹ and yₜ⁰. The ITE analogue in this setting may be referred to as a period treatment effect (PTE), defined as a difference between yₜ¹ and yₜ⁰.

In some cases, PTE is not identifiable due to the fundamental problem of causal inference. However, if X is randomized at every period, then E(Y^{s}), e.g., the mean PO taken across all periods, may be identifiable using observed data by the analogous derivation to Equation 1. Sequential analogues of ignorability and conditional ignorability may be invoked with this time series setting.

In some cases, a difference between E(Y¹) and E(Y⁰) may be referred to as an average period treatment effect. In some cases, it may be taken as the average over the superpopulation comprised of all observed periods 1, . . . , m. In some cases, a broader goal that reflects those of nomothetic causal generalizability or transportability might be used to estimate an APTE taken over all possible relevant periods throughout the participant's life (e.g., the times when they are at risk for condition Y at varying levels of exposure X).

Modifying the earlier example of PO mechanisms, Yₜ¹=β₀+β₁+WₜB₂+εₜ and Yₜ⁰=β₀=Wₜβ₂+εₜ is derived, assuming generally that the PTE is a constant at each period. As with the analogous ATE, the APTE that may be specified as δ^{APTE}=E(δPTE) is also constant and equal to β₁, where the expectation is taken over all periods when X is randomized at every period.

This example reflects the common assumption in n-of-1 trials that the PTEs are all equal to the same constant quantity regardless of period. This property may be referred to as effect constancy; e.g., δ_{y}^{PTE}=β₁. This follows because, for example, all associations between the predictors Xₜ and Wₜ and the observed outcome Yₜ=β₀+β₁Xₜ+εₜ are constant; e.g., the coefficients do not depend on t. Such a predictor-specific association may be described, in some cases, as being stable. This may be a property of n-of-1 trials with no serial interference across periods due to, for example, autocorrelation and carryover of the treatment's influence from past periods.

In some cases, the APTE may be the primary estimand of interest in an n-of-1 trial because all other recurring causes W need not be observed in order to estimate the difference between E(Y₁) and E(Y₀). That is, if X is randomized at each period (e.g., as in a standard n-of-1 trial), then estimates of E(Y|X=s), instead of E(Yₛ), can be used can be used to estimate the APTE without needing to observe W or know how it functionally relates to Y. The key assumption here may be that the PTEs are all identical when there is no serial interference.

However, ignoring or mitigating autocorrelation and carryover in observational (e.g., non-experimental) real-world digital health settings may be infeasible. Accordingly, one may allow for serial interference across periods. Hence, while one may assume that the PTE is constant at each period, one may not assume that the PTEs are all equal to a single APTE.

### Examples of N-of-1 Objectives

In some cases, the systems, the methods, the computer-readable media, and the techniques disclosed herein seek to answer the behavior-change self-tracking question, “What is a possible sustained effect (e.g., APTE) of X on Y, that may be modifiable?” For example, using the systems, the methods, the computer-readable media, and the techniques disclosed herein, a user may seek to answer the question “What is a possible average effect of taking more than my average number of steps per minute on my sleep duration if I keep this up over a week, versus taking fewer than my average number of steps per minute during that same week?” Here, in some cases, APTE may be considered to be sustained once it ceases to change after repeated assignment of the same treatment level over successive periods. This may be a between-period equivalent of within-period effect stability.

In some cases, autocorrelation and carryover can delay an APTE from becoming sustained. The systems, the methods, the computer-readable media, and the techniques disclosed herein may lay the groundwork by answering the question, “What is the APTE of X on Y if we were to randomize X at every period under similar conditions?” This can be answered by expanding the typical n-of-1 trial technique to allow autocorrelation and carryover to influence the APTE. Hence, the systems, the methods, the computer-readable media, and the techniques disclosed herein provide for when X is, in some cases, randomized at every period, which may be referred to as an n-of-1 experiment to distinguish X from the standard constraints enforced in an n-of-1 trial.

### Examples of Temporal Considerations

In some cases, relationships between variables across periods complicate estimation and interpretability of an APTE in ways not commonly encountered in estimating an ATE—even when interference is present. These complications may arise from autocorrelation, time trends, carryover, and slow onset or decay.

Autocorrelation may occur if Yₜ depends on its history up to [symbol] lagged outcomes [symbol] =(L, L², . . . , [symbol] )Yₜ, where L^{k} is the lag operator defined as L^{k}Yₜ=Yₜ₋ₖ. Note that, in some cases, Yₜ need not depend on all lags up to [symbol] . For example, in some cases, Yₜ may depend on Y̅ₜ⁴ only through (L¹, L³, L⁴) or (L², L⁴) for a given mechanism.

Let Vₜ∈Wₜ denote the set of exogenous causes of Yₜ, such that Vₜ is unaffected by X or Y. For example, a temporal cycle like weekday, week of month, month, annual quarter, or season may be included in Vₜ. If all lagged outcomes in [symbol] affect Yₜ, then the structural causal mechanism is Yₜ=g(Xₜ, [symbol] , Vₜ, εₜ). For simplicity of exposition, assume that there are no endogenous causes of Yₜ (e.g., that are affected by Y, and maybe X) from here until discussion on examples of autoregressive carryover models; e.g., assume that Vₜ=Wₜ.

For example, suppose Yₜ=β₀+β_{X}Xₜ+β_{AR}Yₜ₋₁+Vₜβₑₓ+εₜ, where AR stands for “autoregressive.” For such examples of linear models, assume |β_{AR}|<1 for all lagged coefficients; this may be referred to as the stationarity condition in econometrics. Further, in some cases, assume that {(Vₜ)} is jointly covariance stationary; equivalently, that joint weak- or wide-sense stationarity (WSS) of {(Vₜ)} holds. These two conditions may imply that {(Y_{y})} is WSS, enabling meeting of the initial assumption.

In some cases, a time trend may be defined as a sequential trend in the outcomes over successive periods such that E(Yₜ) increases or decreases across periods, rendering {(Yₜ)} no longer WSS. For example, the mean outcome E(Yₜ) may increase with t as with Yₜ=β₀+β_{X}Xₜ+β_{trend}t+Vₜβₑₓεₜ, where β_{trend}>0.

In some cases, carryover may be the tendency for treatment effects to linger beyond the crossover (e.g., when one treatment is stopped and the next one started). Suppose, for example, Yₜ depends on some set of lagged treatments [symbol] =(L, L², . . . , [symbol] )Xₜ. In some cases, if all lagged treatments in [symbol] affect Yₜ, then the structural causal mechanism may be Yₜ=g(Xₜ, [symbol] , Vₜ, εₜ). Such cases may be referred to as having carryover, carryover influence, or treatment carryover.

### Examples of Serial Interference and Effect Modification

In some cases, the presence of carryover or autocorrelation may mean that POs at a given period may be influenced by treatment assignment in past periods. That is, carryover can create serial interference over time, as noted in various randomization-based temporal techniques. Notably, as demonstrated in the following example, carryover does not necessarily modify the PTE itself.

If carryover is present, in some cases, the structural causal mechanism may be Yₜ=g(Xₜ, [symbol] , Vₜ, εₜ). To illustrate, suppose [symbol] =Xₜ₋₁ and Yₜ=β₀+β_{X}Xₜ+β_{co}Xₜ₋₁+Vₜ+εₜ where β_{co}0 at t=1. Let Y^{s}^{ₜ}^{s}^{_{t−1}} denote the potential outcome if the participant receives treatment sₜ at period t, and treatment sₜ₋₁ at period t−1. The PO mechanism may therefore be, in some cases, gₛₛ_{_{t−1}}(Vₜ, εₜ)=β₀+β_{X}sₜ+β_{co}sₜ₋₁+Vₜ+εₜ. In some cases, serial causal consistency states that Yₜ=Yₜ^{X}^{ₜ}^{X}^{_{t−1}}Σ₍ₛ_{ₜ}ₛ_{_{t−1}}₎Yₜ^{s}^{ₜ}^{s}^{_{t−1}}|(Xₜ=sₜ, Xₜ₋₁=sₜ₋₁).

Let Yₜ^{s}^{ₜ}^{⋅} denote the current average PO (CAPO) at period t corresponding to sₜ. In some cases, extending to multiple individuals, this may be referred to as contemporaneous average PO across all individuals at a particular period. This is the potential outcome at period t under treatment level s at that same period, averaged over all possible treatment level combinations over all past periods; in this case, just sₜ₋₁∈{0, 1}. That is, Yₜ^{s}^{ₜ}^{⋅}=E_{X}_{_{t−1}}(Y^{s}^{ₜ}^{X}^{_{t−1}}|Xₜ=sₜ)=Yₜ^{s}^{ₜ}¹Pr(Xₜ₋₁=1|Xₜ=sₜ)+Yₜ^{s}^{ₜ}⁰Pr(Xₜ₋₁=0|Xₜ=sₜ).

Similarly, let Yₜ^{⋅s}^{_{t−1}} denote the carryover average PO at period t corresponding denote the carryover average PO at period t corresponding to sₜ₋₁. This may be the potential outcome at period t under treatment level s at the previous period, t−1, averaged over all possible treatment level combinations in the current period, in this case, sₜ∈{0, 1}. That is, Yₜ^{⋅S}^{_{t−1}}=E_{X}_{ₜ}(Y^{X}^{ₜ}^{s}^{_{t−1}}|Xₜ₋₁=sₜ₋₁)=Pr(Xₜ=1|Xₜ₋₁=sₜ₋₁)+Yₜ^{0s}^{_{t−1}}Pr (Xₜ=0|Xₜ₋₁=sₜ₋₁).

PTE may now be redefined as a difference between the CAPOs Yₜ^{1⋅} and Yₜ^{0⋅}, which may be specified as the difference δₜ^{PTE}=Yₜ^{1⋅}−Yₜ^{0⋅}. Likewise, the carryover effect may be defined as a difference between carryover average POs Yₜ^{⋅1} and Yₜ^{⋅0}, which may be specified as the difference δₜ^{co}=Yₜ^{⋅1}−Yₜ^{⋅0}. In some cases, both differences may be referred to as period average direct causal effects.

Continuing this example, suppose X is randomized to 0 or 1 with equal probability (e.g., all conditional probabilities are equal to 0.5). Table 1 lists the four POs. Accordingly, Yₜ^{1⋅}=Yₜ¹¹0.5+Yₜ¹⁰0.5=β₀+β_{X}+β_{co}0.5+Vₜ+εₜ and Yₜ^{0⋅}=Yₜ⁰¹0.5+Yₜ⁰⁰0.5β₀+β_{co}0.5+Vₜεₜ. Therefore, δₜ^{PTE}=β_{X} at any period t. Note that the PTE may be β_{X} regardless of whether or not a carryover effect (and by implication, carryover) exists, e.g., it is β_{X} both when β_{co}≠0 and β_{co}=0. Hence, neither the carryover effect nor influence modifies the PTE.

TABLE 1

| Yₜ^{StSt-1} | β₀ + β_{X}^{S}ₜ + β_{co}^{S}ₜ₋₁ + Vₜ + εₜ |
|---|---|
| Yₜ¹¹ | β₀ + β_{X} + β_{co} + Vₜ + εₜ |
| Yₜ¹⁰ | β₀ + β_{X} + Vₜ + εₜ |
| Yₜ⁰¹ | β₀ + β_{co} + Vₜ + εₜ |
| Yₜ⁰⁰ | β₀ + Vₜ + εₜ |

Now, suppose instead that Yₜ=β₀+β_{X}Xₜ+β_{co}Xₜ₋₁+β_{Xco}XₜXₜ₋₁+Vₜ+εₜ, where β_{co}=β_{Xco}=0 at t=1, with the four POs listed in Table 2. Therefore, Yₜ^{1⋅}=β₀+β_{X}+β_{co}0.5+β_{Xco}0.5+Vₜ+εₜ and Yₜ⁰=β_{0⋅}+β_{co}0.5+Vₜ+εₜ. Hence, δₜ^{PTE}=β_{X}+β_{Xco}0.5 at any period t. The carryover POs may now be Yₜ^{⋅1}=β₀+β_{X}0.5+β_{co}+β_{Xco}0.5+Vₜ+εₜ and Yₜ^{⋅0}=β₀+β_{X}0.5+Vₜ+εₜ. Hence, δₜ^{co}=β_{co}+β_{X}0.5 at any period t. Note that the PTE may be just β_{X} when there is neither a carryover effect nor influence; e.g., β_{co}=−β_{X}0.5 and β_{co}=0, respectively. However, δₜ^{PTE}=β_{X}−β_{co} when there is no carryover effect, but there is a carryover influence; e.g., β_{co}=−β_{Xco}0.5 but β_{co}≠0, respectively. That is, in some cases, carryover modifies the PTE in general—even when there is no carryover effect.

TABLE 2

| Yₜ^{StSt-1} | β₀ + β_{X}^{S}ₜ + β_{co}^{S}ₜ₋₁ + β_{co}^{S}ₜ₋₁ + Vₜ + εₜ |
|---|---|
| Yₜ¹¹ | β₀ + β_{X} + β_{co} + β_{Xco} + Vₜ + εₜ |
| Yₜ¹⁰ | β₀ + β_{X} + Vₜ + εₜ |
| Yₜ⁰¹ | β₀ + β_{co} + Vₜ + εₜ |
| Yₜ⁰⁰ | β₀ + Vₜ + εₜ |

In some cases, if there is no carryover, autocorrelation can still create serial interference. Recall that the autocorrelation structure causal mechanism is Yₜ=g(Xₜ, [symbol] , Vₜ, εₜ). For example, suppose that [symbol] =Yₜ₋₁, then Y₁=g(X₁, V₁, ε₁) and Yₜ=g(Xₜ, Yₜ₋₁, Vₜ, εₜ) at t>1. Even in this simple example, the resulting POs may undergo a recursive combinatorial expansion with every successive period due to the autocorrelation. Accordingly, estimating an APTE can easily become intractable with more and more periods.

For example, note the sequence of PO mechanisms, Y₁^{s}^{₁}=gₛ_{₁}(V₁, ε₁), Y₂^{s}^{₂}^{s}^{₁}=gₛ_{₂}ₛ_{₂}(Y₁¹₁+Y₁⁰(1−s₁), V₂, ε₂), and Y₃^{s}^{₃}^{s}^{₂}^{s}^{₁}=gₛ_{₃}ₛ_{₁}=(Y₂^{1s}^{₁}s₂+Y₂^{0s}^{₁}(1−s₂), V₃, ε₃). For example, consider our earlier structural causal mechanism, Yₜ=β₀+β_{X}Xₜ+β_{AR}Yₜ₋₁+Vₜβₑₓ+εₜ, where β_{AR}=0 at t=1. Then, Y₁^{s}^{₁}=β₀+β_{X}s₁+βₑₓV₁+ε₁, Y₂^{s}^{₂}^{s}^{₁}=β₀+β_{X}s₂+βₑₓV₂+ε₂, Y₃^{s}^{₃}^{s}^{₂}^{s}^{₁}β₀+β_{X}s₃+β_{AR}Y₂^{s}^{₂}^{s}^{₁}+βₑₓV₃+ε₃, and so on.

### Examples of Average Period Effects

In some cases, the APTE can still be defined in the presence of serial interference. A type of APTE may be introduced based on the past exposure history. Further, the APTE may relate to this history-based quantity through the CAPO.

Let X̅ₜ∈{(L, L², . . . , L^{t-1})}Xₜ={(Xₜ₋₂, Xₜ₋₂, . . . , X₁)} denote the full exposure history where X is first assigned at t=1. A general formula for the historical PTE may be defined as δₜ(x̅ₜ)=Yₜ^{1x̅}^{ₜ}−Yₜ^{0x̅}^{ₜ}. Similar to defining the set of lagged outcomes for autocorrelation, X̅ₜ may not include all past time points. For example, x̅ₜ=xₜ₋₁ at all t in Table 1 and Table 2. The historical PTE in Table 1 may, in some cases, always be δₜ(x̅ₜ)=βₓ. However, Table 2 shows that at t=1, δₜ(x̅ₜ)=βₓ while for t>1 δₜ(1)=βₓ+βₓ_{_{co}} and δₜ(0)=βₓ.

In some cases, historical APTE (HAPTE) may be defined as

$${δ}^{HAPTE} ( \overline{x} ) = \frac{1}{m} {\sum }_{t = 1}^{m} {δ}_{t} ( {\overline{x}}_{t} ) .$$

The HAPTE may be the average effect taken over all periods under the observed exposure history up to m−1; e.g., x̅ₘ. This may answer the question for a subject, “What was the average effect over my particular history of exposures and unchangeable exogenous characteristics (e.g., weather)?” The HAPTE in Table 1 may be always

$${δ}^{HAPTE} ( {\overline{x}}_{m} ) = \frac{1}{m} {\sum }_{t = 1}^{m} {β}_{X} = {β}_{X} .$$

However, in Table 2, HAPTE is defined as

$${δ}^{HAPTE} ( {\overline{x}}_{m} ) = {β}_{X} + I ( m > 1 ) \frac{1}{m} {\sum }_{t = 2}^{m} I ( {x}_{t - 1} = 1 ) {β}_{{X}_{co}} .$$

If there is no carryover, autocorrelation, or any other source of serial interference (e.g., an unobserved exogenous confounder Vₜ₋₂ that affects both Xₜ₋₁ and Yₜ), then the observed exposure history may not influence the POs. Suppose this is the case, such that there is no serial interference, and therefore Yₜ^{s}^{ₜ}^{s̅}^{ₜ}=Yₜ^{s}^{ₜ}. Hence, δₜ(x̅ₜ)=Yₜ¹−Yₜ⁰ as would be the case in Table 1 under βco=0.

Furthermore, suppose that, as in the earlier n-of-1 trial example used to define an APTE, X is randomized at every period, and that the PTEs and APTEs are all equal to the same effect-constant quantity across all periods, denoted δ^{APTE} as before. Then δ^{HAPTE}(x̅ₘ)=δ^{APTE}, so that estimating this trivial APTE is straightforward. Note that this may also be the case in Table 1, showing that the HAPTE may be constant, implying that its expectation may be constant. Accordingly, this n-of-1 trial may answer the question for a subject, “What average effect should I have expected at any given period, if I had randomized intervention at that period?”

However, it should be understood that serial interference that produces non-constant HAPTEs (as in Table 2) cannot always be ruled out, and effect constancy may not hold in general, such that δ^{HAPTE}(x̅ₘ)≠δ^{APTE}. To answer n-of-1 questions like the one above, one cannot always rely on estimating the HAPTE even when X is randomized at every period. Instead, one should generally first define each CAPO used to calculate δₜ^{PTE}=Yₜ^{1⋅}−Yₜ^{0⋅}.

In some cases, the general formula for the CAPO may resemble the logic of causal consistence. The CAPO may accordingly be defined as Yₜ^{s}^{ₜ}^{⋅}=Yₜ^{s}^{ₜ} at t=1, and otherwise,

$$\begin{matrix}{Y}_{t}^{{s}_{{t}^{*}}} = {E}_{{\overline{X}}_{t}} ( {Y}_{t}^{{s}_{t} {\overline{X}}_{t}} \vert {X}_{t} = {s}_{t} ) \\ = {E}_{{\overline{X}}_{t}} \{ {\sum }_{\{ \overline{{s}_{t}} \}} {Y}_{t}^{{s}_{t} {\overline{s}}_{t}} I ( {\overline{X}}_{t} = {\overline{s}}_{t} ) \vert {X}_{t} = {s}_{t} \} \\ = {\sum }_{\{ \overline{{s}_{t}} \}} {Y}_{t}^{{s}_{t} {\overline{s}}_{t}} {E}_{{\overline{X}}_{t}} \{ I ( {\overline{X}}_{t} = {\overline{s}}_{t} ) \vert {X}_{t} = {s}_{t} \} \\ = {\sum }_{\{ \overline{{s}_{t}} \}} {Y}_{t}^{{s}_{t} {\overline{s}}_{t}} {\sum }_{\{ {\overline{x}}_{t} \}} I ( {\overline{X}}_{t} = {s}_{t} ) I ( {\overline{X}}_{t} = {\overline{x}}_{t} ) Pr ( {\overline{X}}_{t} = {\overline{x}}_{t} \vert {X}_{t} = {s}_{t} ) \\ = {\sum }_{\{ \overline{{s}_{t}} \}} {Y}_{t}^{{s}_{t} {\overline{s}}_{t}} Pr ( {\overline{X}}_{t} = {\overline{s}}_{t} \vert {X}_{t} = {s}_{t} )\end{matrix} . \qquad ( Equation 2 )$$

Note that δ_{t PTE} may be similar to the “average contemporary direct effect.” However, whereas the average contemporary direct effect may take the expectation over all individuals at a given period, the expectation in Equation 2 may be taken over all periods prior to t for a single individual.

Suppose X is randomized at every period, reducing the conditional probability component of Equation 2 to Πₛ_{ₖ}_{∈s̅}_{ₜ}Pr(Xₖ=sₖ), and the CAPO to Yₜ^{s}^{ₜ}^{⋅}=E_{x̅}_{ₜ}(Yₜ^{s}^{ₜ}^{X̅}^{ₜ}). Recalling the earlier definition that the expectation E(δ^{PTE}) be taken over all periods when X is randomized at every period in defining δ^{APTE}, yields,

$$\begin{matrix}{δ}^{APTE} = E ( {δ}^{PTE} ) \\ = \frac{1}{m} {\sum }_{t = 1}^{m} {δ}_{t}^{PTE} \\ = \frac{1}{m} {\sum }_{t = 1}^{m} ( {Y}_{t}^{1 *} - {Y}_{t}^{0 *} ) \\ = \frac{1}{m} {\sum }_{t = 1}^{m} \{ {E}_{{\overline{X}}_{t}} ( {Y}_{t}^{1 {\overline{X}}_{t}} ) - {E}_{{\overline{X}}_{t}} ( {Y}_{t}^{0 {\overline{X}}_{t}} ) \} by \\ randomization of {X}_{t} \\ = \frac{1}{m} {\sum }_{t = 1}^{m} \{ {E}_{{\overline{X}}_{t}} ( {Y}_{t}^{1 {\overline{X}}_{t}} - {Y}_{t}^{0 {\overline{X}}_{t}} ) \} \\ = \frac{1}{m} {\sum }_{t = 1}^{m} [ {E}_{{\overline{X}}_{t}} \{ {δ}_{t} ( {\overline{X}}_{t} ) \} ] \\ = {E}_{{\overline{X}}_{t}} \{ \frac{1}{m} {\sum }_{t = 1}^{m} {δ}_{t} ( {\overline{X}}_{t} ) \} \\ = {E}_{{\overline{X}}_{m}} \{ {δ}^{HAPTE} ( {\overline{X}}_{m} ) \}\end{matrix} . \qquad ( Equation 3 )$$

Therefore, in some cases, APTE may be equal to mean HAPTE taken over all possible exposure histories. In some cases, the right side of the second equivalence may be referred to as an average distributional shift effect a generalization of the original group- and population-level quantities.

The n-of-1 experimental quantity of Equation 3 may be referred to as target of inference that answers the question for a subject, “What was the average effect over my particular history of exposures and unchangeable exogenous characteristics, if I had randomized intervention at every period?” This quantity may be estimated using observed data (e.g., without knowing counterfactuals) if X is randomized at every period. To see this, note that the conditional mean observed outcome at a given period is equal to the CAPO.

$$\begin{matrix}E ( {Y}_{t} \vert {X}_{t} = {x}_{t} ) = E ( {Y}_{t}^{{X}_{t} {\overline{X}}_{t}} \vert {X}_{t} = {x}_{t} ) by \\ causal consistency \\ = {E}_{{\overline{X}}_{t}} ( {Y}_{t}^{{x}_{t} {\overline{X}}_{t}} \vert {X}_{t} = {x}_{t} ) \\ = {Y}_{t}^{{x}_{t} \cdot } by Equation 2\end{matrix} . \qquad ( Equation 4 )$$

### Hence,

$${δ}^{APTE} = \frac{1}{m} {\sum }_{t = 1}^{m} \{ E ( {Y}_{t} \vert {X}_{t} = 0 ) \}$$

when X is randomized at every period.

Interestingly, note that the APTE of Equation 3 may actually be a conditional APTE because the original values of the unchangeable exogenous characteristics may be unmodified. This may reflect how in practice, one may not be able to modify such characteristics. Specifically, δ^{APTE}=E(δ^{PTE}|V₁, . . . , Vₘ) where each δₜ^{PTE} depends on the corresponding Vₜ.

Accordingly, in some cases, average PO may be estimated at any period that is stable or constant in the long run (e.g., for very long multivariate time series). Accordingly, if {(Wₜ)} is WSS, a constant long-run APTE may be defined and thereby estimated. In some cases, if such an APTE exists, this property may be referred to as “long-run effect constancy.”

Recall that effect constancy does not require WSS to hold for the average effect to be constant at every period. However, effect constancy may be overly optimistic to expect from real-world observational data. In such cases, long-run effect constancy may still be a reasonable property to expect from a large enough sample (e.g., a long-enough multivariate time series). In some cases, an autoregressive carryover model may imply long-run effect constancy.

### Examples of Autoregressive Carryover Models

In some cases, a basic linear model that accounts for both carryover and autocorrelation, may be an autoregressive carryover (ARCO) model. The ARCO model may be based on dynamic regression models and may enable deriving useful long-run averages (including a long-run APTE) under WSS. The ARCO model may be defined as,

Yₜ=β₀+β_{X}Xₜ+ [symbol] β_{co}+Xₜ^{⊗}β_{X}_{_{co}}+ [symbol] β_{AR}+Yₜ^{⊗}β_{X}_{_{AR}}+Vₜβₑₓ+εₜ

Here, Xₜ^{⊗} may be the vector of all unique two-way interactions (e.g., products) between the elements of (Xₜ [symbol] ), and Yₜ^{⊗} may be the vector of all unique two-way interactions between the elements of (Xₜ, [symbol] ). Equation 5 may be considered both a special case and generalization of a vector autoregressive model; the former because it involves only two three series, one of which is exogenous, and the latter because of the interaction terms.

Note that, in some cases, the ARCO model can be generalized as part of the exponential family by treating its linear component as η, the linear predictor of a generalized linear model (GLM). Specifically, let η_{ARCO}=β₀+β_{X}Xₜ+ [symbol] β_{co}+Xₜ^{⊗}B_{X}_{_{co}}+ [symbol] βₐᵣ+Yₜ^{⊗}β_{X}_{ₐᵣ}+Vₜβₑₓ. Therefore, certain dynamic regression models may be considered a special case of the resulting generalized ARCO model, with a binary outcome and the canonical logit link function. Hence, the structural causal model g(Xₜ, [symbol] , [symbol] , Wₜ, εₜ) may be specified using the ARCO model and {(Wₜ)} may be assumed to be WSS, unless otherwise stated.

### Examples of ARCO in N-of-1 Experiments

In some cases, three long-run averages may be derived via an ARCO model of lag order 1 (here, with [symbol] =Xₜ₋₁ and [symbol] =Yₜ₋₁) when X is randomized at every period as in an n-of-1 experiment. These averages may enable identifying and estimating the APTE directly using this simple model's parameters. However, to fit more flexible models for use in APTE discovery, a model-agnostic technique may be deployed to estimate the APTE. Model-twin randomization may be one such technique.

In some cases, an order-1 model is Yₜ=Yₜ^{X}^{ₜ}^{X}^{_{t−1}}^{X}^{ₜ₌₁}=β₀+β_{X}Xₜ+β_{co}Xₜ₋₁β_{Xco}XₜXₜ₋₁+βₐᵣYₜ₋₁+β_{Xar}XₜXₜ₋₁+Vₜβₑₓ+εₜ. This may be because of an implicit recursive dependence of Yₜ on the exposure history beyond t−1; e.g., Yₜ₋₁ may be a function of Xₜ₋₂ and Yₜ₋₂, and Yₜ₋₂ may be a function of Xₜ₋₃ and Yₜ₋₃, etc.

In general, E(Yₜ|Xₜ=xₜ)=β₀+β_{X}xₜ+β_{co}E(Xₜ₋₁|Xₜ=xₜ)+β_{Xcox}xₜE(Xₜ₋₁|Xₜ=xₜ)+βₐᵣE(Yₜ₋₁|Xₜ=xₜ)+β_{Xar}xₜE(Yₜ₋₁|Xₜ=xₜ)+E(Vₜ|Xₜ=xₜ)βₑₓ. Suppose X is randomized with probability Pr(X=1)=π such that E(Xₜ₋₁|Xₜ=xₜ)=E(Xₜ₋₁)=Pr(Xₜ₋₁=1)=π, E(Yₜ₋₁|Xₜ=xₜ)=E(Yₜ₋₁), and E(Vₜ|Xₜ=xₜ)=E(Vₜ). Furthermore, E(Yₜ₋₁)=E(Y)=μY because {(Yₜ)} is WSS; likewise, E(Vₜ)=μv.

Finally, recall from Equation 4 that E(Yₜ|Xₜ=xₜ)=Yₜ^{x}^{ₜ}^{⋅}. Hence, long-run effect constancy holds because δₜ^{PTE}=Yₜ^{1⋅}−Yₜ^{0⋅}=E(Yₜ|Xₜ=1)−E(Yₜ|Xₜ=0)=β_{X}+β_{Xco}π+β_{Xar}μY is constant across all periods in the long run. Therefore, δ^{APTE}=β_{X}+β_{Xco}π+β_{Xar}μY based at least in part on Equation 3. After re-arranging terms, E(Yₜ|Xₜ=xₜ)=α+xₜδ^{APTE} where α=β₀+β_{Xco}π+β_{Xar}μY+μVβₑₓ.

When the APTE is not modified by carryover such that β_{Xco}=0, and is not modified by past outcomes such that β_{Xar}=0, it may reflect the impact of Xₜ through β_{X}. If carryover's modifying influence is positive such that β_{Xco}>0, this may amplify the impact of Xₜ in proportion to the probability that Xₜ=1; a negative influence dampens its impact. For completeness, in an n-of-1 experiment the long-run mean outcome may be represented by,

$${μ}_{Y} = \frac{{β}_{0} + {β}_{X} π + {β}_{Co} π + {β}_{XCo} {π}^{2} + {μ}_{V} {β}_{ex}}{1 - {β}_{ar} - {β}_{Xar} π} .$$

### Further Examples of ATEs, APTEs, and HAPTEs

In some cases, the HAPTE may be useful to a self-tracker interested primarily in past effects given their past behavior, which may be suitable if the goal is mainly to diagnose what already happened. However, if the self-tracker's goal is treatment or intervention by first understanding the effect or impact in order to change the outcomes by changing their behavior (e.g., not replicate the same exposure history), the HAPTE may not be the appropriate estimand.

The APTE question of “What might the effect be in an n-of-1 experiment?” may be less intuitive than the one posed earlier: “What is a possible sustained effect of X on Y, that I might be able to modify?” However, the importance of questions like “what might the effect be in an n-of-1 experiment” that are answerable by the APTE in accordance with the systems, the methods, the computer-readable media, and the techniques disclosed herein, should be appreciated.

The quantity δ^{APTE} may highlight the important differences in interpretation between the APTE and the ATE. This APTE may be a type of expected average treatment effect where the APTE captures the expected effect of changing a random period's treatment if the current study had been an n-of-1 experiment, with its particular history of unchangeable, exogenous characteristics. Contrast this with the ATE, which is the average effect of changing all participants' treatments from X=1 to X=0. Applying this technique to time series data may include, in some cases, assigning X=1 and X=0 at all periods, and comparing the predicted mean potential outcomes under each treatment level. This ATE-type procedure may help answer a related but different question: “What would have been the average effect of changing the treatment to the same level over all periods if the current study had been an n-of-1 experiment, with its particular history of unchangeable, exogenous characteristics?”

This key distinction may foreshadow how the model-twin randomization procedure estimates the APTE, compared to how the analogous ATE-type procedure above works. Briefly, in some cases, model-twin randomization may be a Monte Carlo technique that randomly shuffles the exposure vector over all periods, predicts potential outcomes under each treatment level, adds random noise for better statistical comparison, compares the average predicted noisy outcomes under each treatment level, and then repeats this procedure multiple times until convergence. In contrast, the ATE-type procedure above may just simply change all treatments to 1 or 0 in order to compare outcomes (possibly with noise added).

### Examples of Other Temporal Considerations

In some cases, in an n-of-1 trial, carryover might be avoided in PTE estimation by using a washout period that allows the lingering influence of past treatment to “wash out” of the participant's system. This may be done by design, through analytic adjustment, or both. The physical or design-based washout technique may include not administering the next treatment assignment until the influence of the previous treatment assignment has subsided. The analytic washout technique may include down-weighting or dropping time points at which carryover still exists.

In some cases, an example of either technique may include dropping time points, including t at which Xₜ₋₁=0 (assuming X=0 denotes the baseline treatment at which the outcome is at a baseline level). In some cases, this may constrain the PTE to just Yₜ¹⁰−Yₜ⁰⁰=β_{X}, regardless of whether carryover modifies the overall PTE that allows for carryover. In some cases, one may assume that one cannot designate a washout period a priori, and so one may instead include any treatment history with potential carryover in the models.

In some cases, if a participant is measured multiple times during a period or phase such that j∈{1, . . . , mₜ}, for repeated measurements per period t, there is an opportunity to assess and adjust for slow onset or decay of the treatment effect. For example, a treatment with slow onset may take time to reach its maximum or stable PTE, which may span more than one period. Similarly, a treatment with slow decay may take time to dissipate or wash out. If its washout time spans more than one period, then carryover may still exist. Multiple measurements per phase may be standard practice in SCEDs. In studies that define a day as a period, intraday sensor measurements (e.g., with a Fitbit® or smart watch) may be taken over uniform time intervals commonly called epochs.

### Examples of Estimation Techniques

In some cases, the systems, the methods, the computer-readable media, and the techniques disclosed herein may be applied to n-of-1 trials. For example, in an n-of-1 trial, randomization may eliminate confounding due to autocorrelation and carryover. There may be no such guarantee in an n-of-1 observational study; thus, the possibility of confounding cannot be ignored. There may be a number of techniques existing to address or adjust for confounding in nomothetic studies, thereby estimating the ATE. Such techniques may be adapted for estimating the APTE; for example, a g-formula technique may directly adjust for autocorrelation-induced and carryover-induced confounding.

Recall that to estimate the ATE, E(Y^{x}) can be estimated directly if X is randomized by estimating E(Y|X=x) using only observed values. In some cases, directly modeling the outcome mechanism Y=g(X, W, E) need not use an outcome model. This may follow from the law of total expectation,

$$\begin{matrix}E ( Y \vert X = x ) = {E}_{w} \{ E ( Y \vert X = x , W ) \vert X = x \} \\ = {E}_{w} \{ E ( Y \vert X = x , W ) \} if \\ X is randomized \\ = E ( {Y}^{x} ) by ( 1 )\end{matrix} . \qquad ( Equation 6 )$$

Specifically, this result may explain why E(Y^{x}) can be inferred from the estimate of E(Y|X=x) when X is randomized, without having to model E(Y|X=x, W). Nonetheless, an outcome model may still be fit to correctly estimate the ATE (e g., using an asymptotically consistent estimator).

First, note that fitting an outcome model may be a ubiquitous practice in both statistical and machine learning settings; explicitly in the former, implicitly in the latter as the prediction function Y˜f(X, W). Due at least in part on causal consistency, the outcome mechanism may be related to the PO mechanism as Y=g(X, W, E)=Σ_{{s}}gₛ(W, E)I(X=s), with shorthand Y=g_{X}(W, ε). Hence, the outcome mechanism g(X, W , ε) or the PO mechanism g_{X}(W, ε) may be modeled together as both may be mathematically identical (e.g., specifically, for a given value of X).

In some cases, fitting the correct outcome model to the observed values may enable estimating the conditional mean E(Y|X=s, W)=Eₛ(g(X, W, ε)|X=s, W) for each exposure level s. To illustrate, recall that g(X, W, ε)=β₀+β₁X+Wβ₂+ε. Estimating the parameters {β₀, β₁, β₂} to estimate E(Y|X=s, W) may enable, for each s∈{0, 1}, predicting outcome values as Ŷᵢ^{s} for all study participants i=1, . . . , n. Following Equation 6, E(Y^{s}) may be estimated by averaging over all predicted values for each s∈{0, 1}; e.g.,

$${\overline{\hat{Y}}}^{s} = \frac{1}{n} {\sum }_{i = 1}^{n} {\hat{Y}}_{i}^{s} .$$

Finally, ATE may be estimated as the difference between these averages; e.g., \hat{Y̅}¹−\hat{Y̅}⁰.

However, in some cases, this procedure may also work for estimating E(Yₓ) even if X is not randomized. This result serves as a key insight underlying the technique of g-formula or g-computation formula, which may also be referred to as direct standardization, stratification, regression adjustment, or the back-door adjustment formula. The g-formula may be the equivalence of the penultimate and last expressions of Equation 6. The g-formula states that if one has observed all confounders and knows the outcome mechanism, then one can correctly estimate the ATE by replicating the probability conditions for the corresponding hypothetical RCT.

In some cases, it may be possible or desired to apply this equivalence when X is not randomized. For example, Let R=1 denote the case when X is randomized, and R=0 otherwise. Randomization invariance may hold when g(X, W, R, ε)=g(X, W, ε) (e.g., data-generation invariance) and W [symbol] R (e.g., distributional invariance). These two conditions together imply

$$\begin{matrix}{E}_{W} \{ E ( Y \vert X = x , W ) \} = {E}_{w} \{ E ( Y \vert X = x , W < R = 0 ) \vert R = 0 \} \\ = {E}_{w} \{ E ( Y \vert X = x , W , R = 1 ) \vert R = 1 \} \\ = {E}_{w} \{ E ( Y \vert X = x , W ) \} if X is randomized \\ = E ( {Y}^{*} ) by Equation 1\end{matrix} .$$

Hence, the g-formula may be applicable, in some cases, if randomization invariance holds Further, it may be noted that randomization of X may be a particular way to set or fix X, such that p(y|do(x))=p(y|X=x, R=1) for the do operator.

In some cases, it may be possible to specify a useful propensity model for Pr(X=s|W), as in, the propensity of seeing X=s for a given level of W. In some cases, a variety of propensity score techniques may be applied to estimate the ATE. For example, these may include the matching and IPW techniques disclosed herein with respect to PO For example, with this technique, X=I(ε^{X}<Pr(X=1|W)), where ε^{X} is uniformly distributed between 0 and 1.

In some cases, it may be useful to characterize the performance (e.g., with respect to empirical bias) of a g-formula estimation technique. However, a complementary propensity-score IPW technique may also be applicable, when used for comparison. Moreover, in some cases, doubly robust techniques (e.g., “augmented IPW”), which involve specifying both outcome and propensity models may be applicable.

### Examples of Modeling Flexibility

In some cases, the outcome and propensity mechanisms may be either unknown or cannot otherwise be reasonably justified (e.g., due to a lack accumulated theoretical evidence). This may be the case for settings of causal hypothesis generation and causal discovery. In such cases, a key objective may include identifying, selecting, or proposing plausible models for a small set of posited causal effects, rather than estimating effects and associations posited by scientifically defensible or otherwise well-established a priori models.

Note that the outcome mechanism in Equation 6 can take any appropriate functional form. For example, these have traditionally been modeled using linearized parametric or semi-parametric models in the statistics literature. However, Equation 6 may apply to the true mechanism, which may or may not be a linearized model. Hence, supervised learning techniques may be applied to allow fitting models focused on characterizing the relationship between X, the exposure of interest, and the outcome Y, while also flexibly accommodating non-exposure variables W. Likewise, the same may hold for modeling the propensity of X.

In some cases, the decision tree may be one such non-parametric technique for estimating a conditional ATE for a continuous outcome (e.g., specified as a difference between mean POs). The decision tree-based technique may be used to estimate an APTE if conditional WSS holds (e.g., the outcomes are WSS conditional on the causes). WSS may replace the assumption of conditional independence (e.g., the outcomes are mutually independent conditional on the causes) used for consistent effect estimation. Unlike linearized models that use prespecification of interaction terms, the decision tree-based technique known as random forests (RF) may be able to implicitly allow for multiple interactions between predictors in predicting the outcome.

In some cases, the systems, the methods, the computer-readable media, and the techniques disclosed herein apply a Single Tree technique (e.g., use of a single decision tree in the implementation of RF involving many decision trees), and model the outcome as a function of both the exposure and other causes using RF. In some cases, the systems, the methods, the computer-readable media, and the techniques disclosed herein may apply a Two Trees technique for conducting feature selection (e.g., for non-exposure causes important for predicting potential outcomes), which involves fitting separate outcome models for each exposure level. Furthermore, the systems, the methods, the computer-readable media, and the techniques disclosed herein may model the exposure propensity directly (e.g., versus a Transformed Outcome Tree technique).

### Examples of Model-Twin Randomization

As previously discussed, a sequential technique may be useful for implementing the g-formula for estimating the APTE. Recall Yₜ may be sequentially generated, unlike the outcome Yᵢ that may be generated across participants in ATE-focused settings.

In some cases, the systems, the methods, the computer-readable media, and the techniques disclosed herein may apply sequential techniques for numerical estimation called model-twin randomization (MoTR). In some cases, MoTR may randomize originally observed sequential exposure {(xₜ)}, thereby changing the originally observed sequential exposure from a possibly endogenous variable (e.g., one that may be affected by past values of Y) into an exogenous variable (e.g., one that may be unaffected by the outcome). Hence, MoTR may be a Monte Carlo technique that provides numerically approximate calculations of statistically consistent estimates of the APTE, along approximate confidence intervals (CIs) for conducting inference.

In some cases, the systems, the methods, the computer-readable media, and the techniques disclosed herein may implement one or more operations, including:
(1) fitting an outcome model, μₜ=E(Yₜ|Xₜ, Wₜ), to observed data, {(xₜ), (wₜ)}, where an estimator, \hat{μ}ₜ, from fitting the outcome model is, for a participant, a model-twin (e.g., a digital twin that represents the participant's outcome mechanism for estimating APTE);
(2) running the model-twin through a simulated n-of-1 trial by performing, for each iteration, r, operations including:
   (a) randomly permuting or shuffling all observed xₜ, where Xᵣ=(Xᵣ₁, . . . , Xᵣₘ) represents this randomized sequence, where this operation may preserve the original ratio of exposures to non-exposures, reflecting the exposure's observed overall propensity;
   (b) generating Wᵣₜ at each period t sequentially, by performing operations including:
      (i) keeping any exogeneous variable, vₜ, contained in wr equal to their corresponding values in Wᵣₜ,
      (ii) setting all lags of Yₜ included in Wₜ to their values generated in past runs (e.g., set Yₜ₋₁ equal to Ŷᵣ₍ₜ₋₁₎, as defined in operation (c)); and
      (iii) setting all lags of Xₜ included in Wₜ to their corresponding values in Xᵣ;
   (c) generating Yᵣₜ at each period t sequentially, by performing operations including:
      (i) predicting an outcome as \hat{μ}ᵣₜ=Ê(Yₜ|Xₜ=Xᵣₜ, Wₜ=Wᵣₜ); and
      (ii) adding random noise to each predicted value as Ŷᵣₜ=\hat{μ}ᵣₜ+εᵣₜ where εᵣₜ˜N(0, σ_{ε}), where σ_{ε} is the standard deviation of the residuals ({Yₜ−\hat{μ}ₜ}), treated as fixed, where \hat{μ}ₜ=Ê(Yₜ|Xₜ=xₜ, Wₜ=wₜ) depends on the observed data {(xₜ), (wₜ)} rather than {(Xᵣₜ), (Wᵣₜ)} (e.g., the data resulting from the random permutation or shuffling of {(xₜ)}), where this operation may preserve summary information about residual variation in the outcome (e.g., as is done to create prediction intervals in statistics), and where Ŷᵣₜ may be an element of W_{rt′}for some future period t′<t;
   (d) estimating E(Yₛ), the mean PO, for s∈{0, 1}, to be the average of the noisy predicted outcomes, e.g.,

$${\overline{\hat{Y}}}_{r}^{s} = \frac{1}{{m}_{s}} {\sum }_{t = 1}^{m} {\hat{Y}}_{rt} I ( {X}_{rt} = s )$$

where mₛΣₜ₌₁^{m}I(Xₜ=s), which may be constant regardless of r, and APTE may be estimated as \hat{σ}ᵣ^{MoTR}=\hat{Y̅}ᵣ¹−Y\hat{Y̅}ᵣ⁰;
(e) estimating corresponding confidence intervals likewise, e.g., via a common t−test if the predicted outcomes are fairly normally distributed; and
   (f) calculating the cumulative average APTE over all previous runs as the average of all estimates δᵣ^{MOTR} up to and including the current run, r, e.g.,

$${\hat{δ}}_{r}^{MoTR} = \frac{1}{r} {\sum }_{h = 1}^{r} {\hat{δ}}_{h}^{MoTR} ,$$

and where the cumulative average confidence intervals are likewise calculated;
(3) performing operations 2(a)-2(e) repeatedly until all three cumulative averages converge based at least in part on reasonable criteria, where to ensure some stability in cumulative estimates, a minimum rₘᵢₙ may be set such that rₘᵢₘ≤rₘₐₓ; and
(4) reporting final values from operation 3 as the APTE estimate \hat{δ}^{MoTR} along with a corresponding confidence interval.

In some cases, any number of operations of the one or more operations described above may be added or removed. Further, the one or more operations described above may be performed in any order. Further, at least one of the one or more operations described above may be repeated, e.g., iteratively.

### Examples of Propensity Score Twins

In some cases, one or more IPW techniques may be implemented by the systems, the methods, the computer-readable media, and the techniques disclosed herein to complement the MoTR techniques. The one or more IPW techniques may be referred to as propensity score twin (PSTn). Unlike MoTR, PSTn may not be a sequential Monte Carlo technique. Instead, PSTn may predict the exposure probability at each period once after modeling the probability of exposure; e.g., Pr(Xₜ=s|Wₜ). In some cases, the systems, the methods, the computer-readable media, and the techniques disclosed herein may apply to characterization bias in PSTn estimation, rather than characterizing inference (e.g., which includes estimating CIs).

In some cases, the systems, the methods, the computer-readable media, and the techniques disclosed herein may implement one or more operations, including:
(1) fitting a propensity model πₜ=Pr(Xₜ=1|Wₜ) to data, where similar to the one or more operations described with respect to MoTR, the estimator, \hat{π}ₜ, from fitting the propensity model may be, for a participant, a propensity score twin (e.g., a digital twin that represents the participant's exposure mechanism for estimating APTE),
(2) weighting each observed Yₜ by the reciprocal of its corresponding estimated propensity based at least in part on observed exposure, e.g., \tilde{Y}ₜ=Yₜ/{xₜ\hat{π}ₜ+(1−xₜ)(1−\hat{π}ₜ) }, where optionally, mitigating overly large weights resulting from overly small estimated properties, including performing operations, may involve performing operations including:
   (a) using trimmed \hat{π}ₜ values (e.g., dropping extreme \hat{π}ₜ, values);
   (b) using overlapping \hat{π}ₜ values (e.g., keeping the range of \hat{π}ₜ values with the most overlap between groups with s∈{0,1}); and
   (c) using stabilized weights (e.g., multiply \tilde{Y}ₜ by the proportion of the sample with X=xₜ for the corresponding values of xₜ;
(3) estimating, for s∈{0,1}), E(Y^{s}) as the average of the weighted outcomes e.g.,

$$= \frac{1}{m} {\sum }_{t = 1}^{m} {\tilde{Y}}_{t} I ( {X}_{t} = s ) ;$$

and
(4) estimating the APTE as \hat{δ}^{PSTn}=

[symbol]

In some cases, any number of operations of the one or more operations described above may be added or removed. Further, the one or more operations described above may be performed in any order. Further, at least one of the one or more operations described above may be repeated, e.g., iteratively.

### Examples of Simulation Studies

In some cases, the systems, the methods, the computer-readable media, and the techniques disclosed herein may use simulated data to characterize the empirical bias of the MoTR and PSTn estimators (\hat{δ}^{MOTR} and \hat{δ}^{PSTn}, respectively). The systems, the methods, the computer-readable media, and the techniques disclosed herein may characterize this bias when each model is correctly specified as the true data-generating mechanism, which may be specified as a GLM (e.g., ARCO for the outcome, logistic for the exposure). The systems, the methods, the computer-readable media, and the techniques disclosed herein may also characterize this bias using naive APTE estimation methods, for example, when no modeling is done, and when the true models are fit but are not adjusted for confounding by either method. The systems, the methods, the computer-readable media, and the techniques disclosed herein may also characterize the empirical bias of the RF techniques disclosed herein with respect to modeling flexibility For example, in some cases, while RF may be more flexible than linearized models in certain useful ways, RF may be more biased because the relevant prediction function is not identical to the true GLM mechanism.

### Examples of Simulation Study Data-Obtaining Procedures

In some cases, data may be collected using one or more of the techniques disclosed herein with respect to FIG. 1. For example, data may be collected using smart clothing or other body-tracking devices or software.

For a specific example to be discussed, 222 days of days of sleep duration and step-count data was collected using a Fitbit® Charge 3 wrist−worn sensor. Hence, each simulated (e.g., simulated) dataset was generated with m=222 time points.

For the specific example, outcome and exposure of interest was specified as follows. A period was specified as a day. Outcome was daily total sleep time (TST), defined as the total number of hours asleep per night. Physical-activity exposure was the daily number of steps per minute awake (chosen, e.g., rather than daily raw step count) to understand if increasing physical activity overall, regardless of how much time the participant was awake, led to longer or shorter sleep; however, simply getting more sleep leaves one less time to take steps the following day). Raw step count may be associated with sleep duration because simply getting more sleep leaves one less time to take steps the following day. Raw step count may simultaneously not affect sleep duration, which is the APTE of interest in the specific example.

In the standard potential outcomes framework, it may be reasonable to dichotomize continuous exposures in order to define clear causal effects between distinct groups or levels. Hence, for the specific example, exposure was dichotomized into high and low physical activity based on the observed median steps per minute over all 222 days of observation.

In the specific example, each simulated dataset (indexed h) was generated using the following operations, which may induce autoregression and confounding via endogeneity (e.g., as might be present in real data).
(1) if t=1, generate Yₕₜ as a continuous variable drawn from β₀+εₜ where εₜ˜N(0, δ_{ε}), otherwise, proceed to operation (2);
(2) for both s=0 and s=1, generate Yₕₜ^{s} using the ARCO PO mechanism Yₕₜ^{s}=β₀+β_{X}s+β_{AR}Yₜ₋₁+εₜ, where Yₜ₋₁ is set equal to Yₕ₍ₜ₋₁₎;
(3) if t=1, generate Xₕₜ∈{0, 1} as a binomial variable with probability π₁=Pr(X₁=1), otherwise, generate Xₕₜ using the propensity mechanism logit(πₜ)=α₀+αₑₙYₜ₋₁ where πₜ=Pr(Xt=1|Yₜ₋₁), and where Yₜ₋₁ is set equal to Yₕ₍ₜ₋₁₎, and where αₑₙ≠0 induces endogeneity by making Xₜ depend on Yₜ₋₁; and
(4) generate the observed outcome Yₕₜ using causal consistency e.g., generate Yₕₜ=Yₜ¹Xₜ+Yₜ⁰(1−Xₜ), where Yₜ^{s} is set equal to Yₕₜ^{s} for both s=0 and s=1, and where Xₜ is set equal to Xₕₜ.

In some cases, any number of operations of the one or more operations described above may be added or removed. Further, the one or more operations described above may be performed in any order. Further, at least one of the one or more operations described above may be repeated, e.g., iteratively.

Note that, parameters used with the specific example include: β₀=2, δ_{ε}=0.5, β_{X}=1.1, β_{AR}=0.8, π₁=0.5, α₀=−0.25, and αₑₙ=1.25. Note that You may be stationary because |β_{AR}|≤1; hence the true APTE was β_{X}=1.1.

### Examples of Analysis Techniques and Results

For the specific example, six estimation techniques were used, as listed in Table 3. Specifically, r=200 MoTR runs were used, and applied trimming and overlapping were used with propensity scores (e.g., which may be stabilized). The coefficient estimate technique was included as an extra coding check to confirm that code was correct (e.g., to confirm expectation of being unbiased over many simulations).

TABLE 3

| | | | Bias |
|---|---|---|---|
| Method | Estimator | Description | Expected |
| Raw comparison of averages (no modelling) | \hat{δ}^{raw} | $\frac{1}{{m}_{1}} {\sum }_{t = 1}^{m} {Y}_{t} I ( {X}_{t} = 1 ) - \frac{1}{{m}_{o}} {\sum }_{t = 1}^{m} {Y}_{t} I ( {X}_{t} = 0 )$ | yes |
| Coefficient estimate | \hat{β}_{X} | GLM estimator of ARCO exposure | no |
| | | coefficient | |
| MoTR-GLM | \hat{δ}^{MoTR-GLM} | \hat{δ}^{MoTR} after fitting GLM outcome model | no |
| PSTn (Stabilized)- | \hat{δ}^{PSTn-GLM} | \hat{δ}^{PSTn} after fitting GLM propensity model | no |
| GLM | | | |
| MoTR-RF | \hat{δ}^{MoTR-RF} | \hat{δ}^{MoTR} after fitting RF prediction function | some |
| PSTn-RF | \hat{δ}^{PSTn-RF} | \hat{δ}^{PSTn} after fitting RF prediction function | some |

The coefficient estimate, MoTR-GLM, and PSTn (Stabilized)-GLM techniques all may include fitting the correct models (e.g., the correct ARCO-outcome or exposure mechanisms). With the specific example, for both the coefficient and MoTR-GLM methods, the outcome model Yₜ=β₀+β_{X}Xₜ+β_{AR}Yₜ₋₁+εₜ was fit where ε˜N(0, δ_{ε}). Further, for the PSTn (Stabilized)-GLM technique, the propensity model logit(πₜ)=α₀+αₑₙYₜ₋₁ where πₜ=Pr(Xₜ=1|Yₜ₋₁) was fit. In some cases, the RF techniques include fitting prediction functions. For the MoTR-RF techniques, the prediction function Yᵣ˜f(Xₜ, Yₜ₋₁) may be fit. For the PSTn-RF technique, the classification function Xₜ˜f(Yₜ₋₁) may be fit.

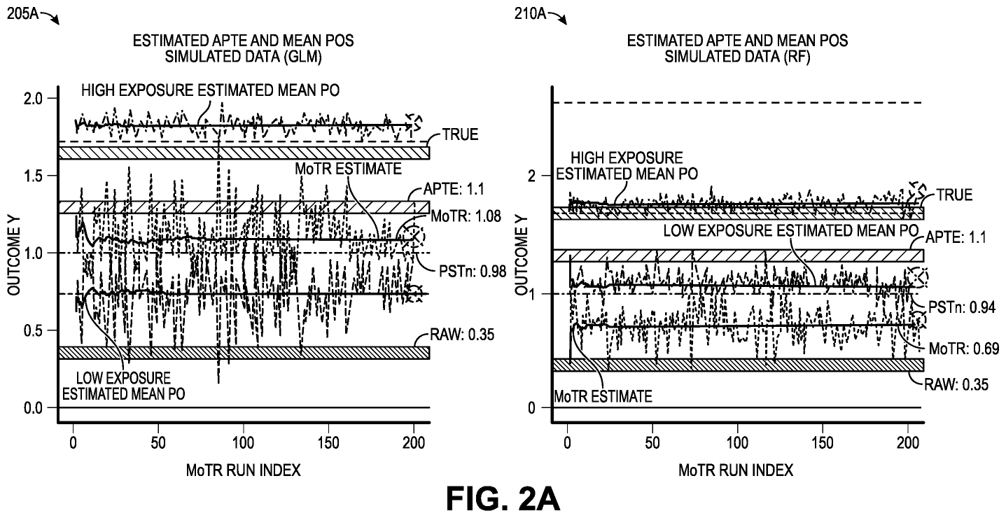

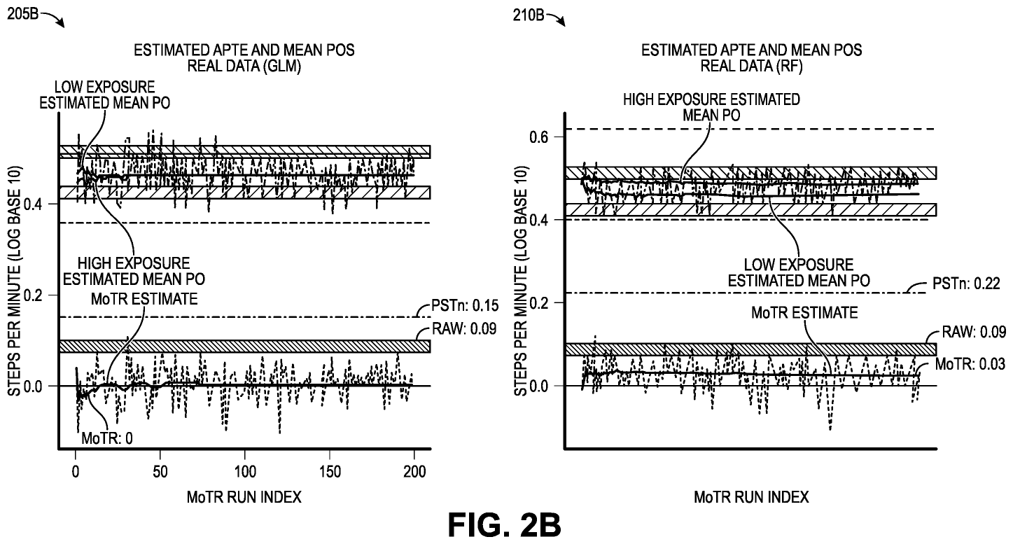

FIGS. 2A and 2B show examples of results of a simulation applying model-twin randomization and propensity score twin.

FIG. 2A specifically shows, for the specific example, the results of analyzing one simulated dataset (h=1) with how the MoTR and PSTn techniques relate to each other. Here, the corrected models were used for MoTR and PSTn, with a sample size of m=220. The corresponding values of each final estimate at run r=200 are shown in Table 4. FIG. 2A shows analysis results of applying each of the MoTR and PSTn techniques to one simulated dataset. For example, 205A illustrates results for a generalized linear model (GLM) and 210A illustrates results for random forests (RF). As illustrated, MoTR used rₘᵢₙ=10 runs to rₘₐₓ=200 runs.

TABLE 4

| Method | Estimate (95% CI) |
|---|---|
| Raw comparison | 0.35 (0.1, 0.6) |
| Coefficient estimate | 1.10 (0.92, 1.27) |
| MoTR-GLM | 1.08 (0.68, 1.48) |
| PSTn (Stabilized)-GLM | 0.98 |
| MoTR-RF | 0.69 (0.4, 0.98) |
| PSTn-RF | 0.94 |

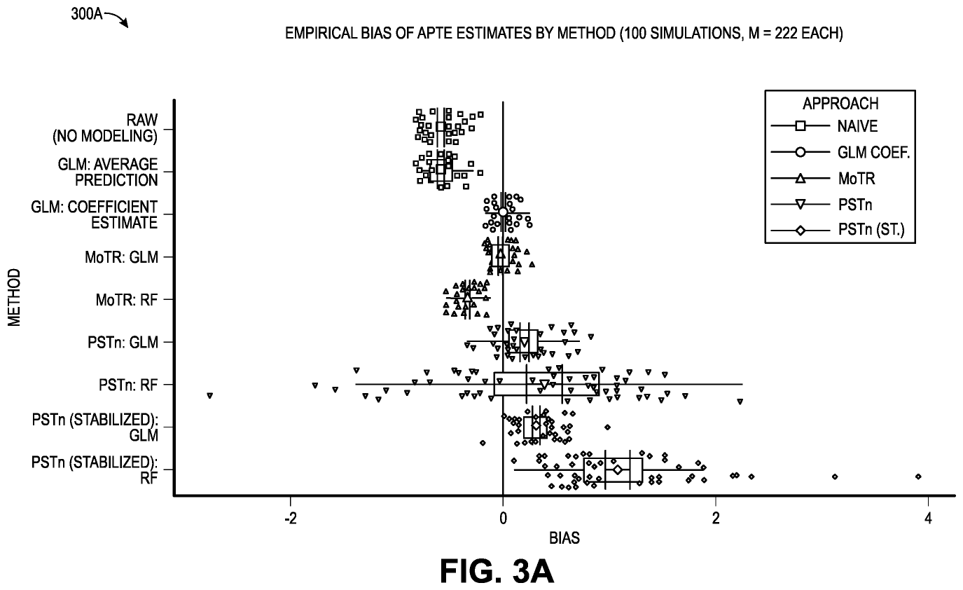

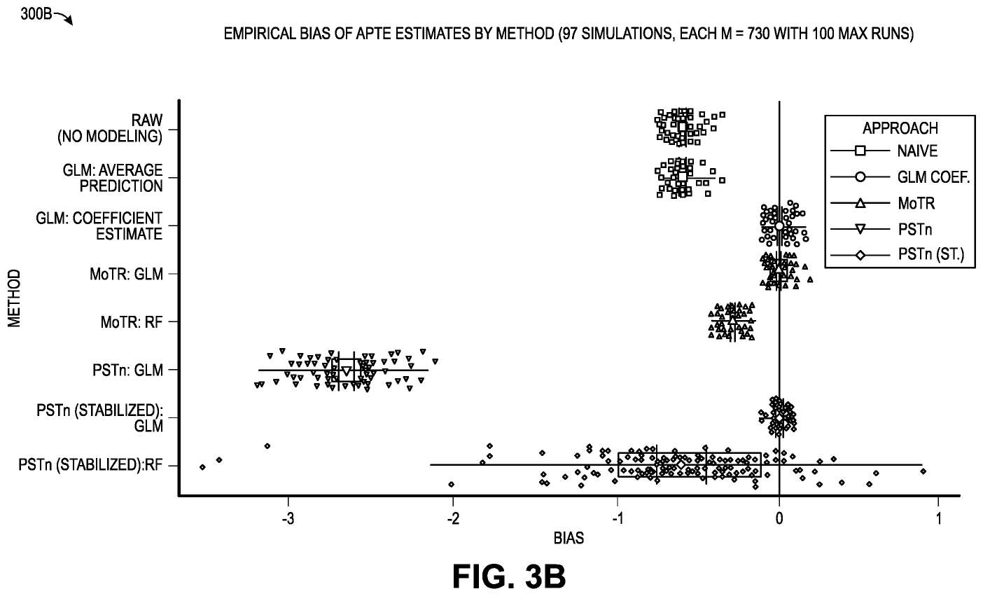

For this particular dataset used with the specific example, the MoTR and PSTn techniques estimate the true APTE with little bias compared to the raw comparison when their models are specified correctly. The MoTR CI even covers the true APTE. However, the MoTR technique that uses RF may be more biased than the corresponding PSTn technique. This result may be due to sample-to-sample variation, as seen in the empirical bias results over 100 simulated datasets shown in FIG. 3A. These techniques may be a way of approximating the true bias of an estimator at a given sample size.

In FIG. 3B, the sample size is increased to m=730. This enables beginning to assess the asymptotic trend in bias of each estimator implied by the central limit theorem (CLT). FIG. 3B shows that all estimators exhibit similar or smaller bias compared to the smaller sample of m=222. In particular, PSTn (Stabilized)-GLM performs better (e.g., with reduced empirical bias), as expected under the CLT.

FIG. 2B specifically shows analysis results of applying each of the MoTR and PSTn techniques to real datasets. For example, 205B illustrates results for a GLM and 210B illustrates results for RFs. As illustrated, MoTR used rₘᵢₙ=10 runs to tₘₐₓ=200 runs.

The notation of FIG. 2B matches that of FIG. 2A. For FIGS. 2A and 2B, one of the solid black lines represents the MoTR estimate (marked as “MoTR: ______”) (e.g., cumulative over all previous APTE estimates) and the non-horizontal dashed black lines behind this solid black line represents each MoTR run's APTE estimate. For FIGS. 2A and 2B. For FIGS. 2A and 2B, one of the dashed black lines represents the PSTn estimate (marked as “PSTn: ______”). For FIGS. 2A and 2B, the thick line represents the raw comparison estimate (marked as “RAW: ______”). For FIGS. 2A and 2B, one of the solid lines represents each of the estimated mean potential outcomes (POs) under high (marked as “HIGH EXPOSURE ESTIMATED MEAN PO”) and low (marked as “HIGH EXPOSURE ESTIMATED MEAN PO”) exposure levels, respectively, for each of the three estimators. Note that the difference between the solid lines of the high and low exposure levels equals the corresponding MoTR estimate for any run. For FIGS. 2A and 2B, the circled X's mark the true mean PO and APTE values.

FIG. 3A shows examples of results of 100 simulated datasets. FIG. 3A shows, with respect to the specific example, for over 100 datasets, that the empirical bias for the raw comparison technique is the largest compared to the coefficient estimate, MoTR-GLM, and PSTn (Stabilized)-GLM techniques, while PSTn (Stabilized)-GLM performs worse than MoTR-GLM. The coefficient estimate technique is unbiased, as expected, confirming that the modeling code was correct. For the RF-based methods, the MoTR-RF performs better than raw comparison. However, MoTR-RF performs worse than MoTR-GLM, as expected. Likewise holds for PSTn-RF, which also is less consistent in that its results vary widely more from sample to sample, as evidenced by its wider spread of points and CI.

Specifically, FIG. 3A illustrates analysis 300A of the results of applying each technique to 100 simulated datasets each with m=220. Each light dot of FIG. 3A represents the empirical bias of one simulated dataset for estimating a true APTE of 1.1 Each dark dot of FIG. 3A represents the average empirical bias over all 100 datasets, with corresponding 95% confidence interval shown as symmetric error bars.

Specifically, FIG. 3B illustrates analysis 300B of the results of applying each technique to 97 simulated datasets with m=730. Each light dot of FIG. 3B represents the empirical bias of one simulated dataset for estimating a true APTE of 1.1. Each dark dot of FIG. 3B represents the average empirical bias over all 100 datasets, with corresponding 95% confidence interval shown as symmetric error bars.

### Examples of Empirical Studies

The systems, the methods, the computer-readable media, and the techniques disclosed herein may be applied to estimate an APTE of nightly TST on steps per minute the next day for a participant. The models of the systems, the methods, the computer-readable media, and the techniques disclosed herein, may include an indicator for various time-related factors, e.g., weekend (versus weekday), which may be treated as an exogenous variable. To reflect the fact that, in practice, when designing a study one cannot modify the day of the week in an n-of-1 trial, the models may not modify original values. Hence, the estimand was that of Equation 3, a conditional APTE.

As with the simulation study described with respect to FIGS. 2A-3B, the models analyzed m=222 days of the participant's sleep duration (an example of time series data relating to health behavior) and step-count data (an example of time series data relating to health condition) using a Fitbit® Charge 3 wrist−worn sensor, collected from May 2019 through December 2020. Then, the time series of the health condition may be transformed from the raw outcome, steps per minute the next day, by taking its log (base 10); this was the outcome analyzed. This log transform may be performed to ensure the outcomes were fairly normally distributed, to meet the GLM modeling assumption of normally distributed residuals. Exposure, nightly TST, may be dichotomized (as was done here), based on the observed median over all 222 days of observation of 7.1 hours; e.g., high TST corresponded to >7.1 hours of sleep on a given night, and otherwise the exposure was labeled as low TST.

The same or similar six estimation techniques listed in Table 3 may be used in this case, except for the coefficient estimate technique (which, e.g., may be used to check that the modeling code is correct). As with this simulation study, r=200 MoTR runs may be performed, and trimming and overlapping may be applied to the stabilized propensity scores. For all four techniques, Wₜ including the endogenous variables Xₜ₋₁ and Yₜ₋₁ (or Qₜ₋₁, explained below), and the exogenous variable Vₜ where Vₜ=1 if day t is a weekend (and Vₜ=0 otherwise).

For this empirical study, the systems, the methods, the computer-readable media, and the techniques disclosed herein may apply various MoTR models. For the MoTR-GLM technique, the outcome model Yₜ=β₀+β_{X}Xₜ+β_{co}Xₜ₋₁+Qₜ₋₁β_{AR}+βₑₓVₜ+εₜ may be fit where εₜ˜N(0, δ_{ε}). Here, Qₜ₋₁ may be a 1×4 vector of zeros and ones indicating the quartile of ({Yₜ)} that Yₜ₋₁ fell into (along with conformable coefficient 4×1 vector B_{AR}. For example, Qₜ₋₁=(0, 0, 1, 0) if Yₜ₋₁ is in the third quartile. This may allow for a more flexible non-linear relationship between the current and lagged outcome, at, ef the expense of losing continuous-valued predictor information. For the MoTR-RF technique, the prediction function Yᵣ˜f(Xₜ, Xₜ₋₁, Yₜ₋₁, Vₜ) may be fit. Note that Yₜ₋₁ may be used instead of Qₜ₋₁ because RF may automatically segment or partition these continuous values, precluding the use of doing so manually. In this empirical example, MoTR was performed using rₘᵢₙ=200 runs and performance over all the runs is illustrated in FIG. 2B.

Further, in this empirical example, various PSTn models may be fit. For the PSTn (Stabilized)-GLM technique the propensity model, logit(πₜ)=α₀+α_{AR}Xₜ₋₁+αₑₙQₜ₋₁+αₑₓVₜ may be fit where πₜ=Pr(Xₜ=1|Xₜ₋₁, Qₜ₋₁, Vₜ). For the PSTn-RF technique, the classification function, Xᵣ˜f(Xₜ₋₁, Yₜ₋₁, Vₜ) may be fit.

The results of these techniques applied to the empirical example in accordance with the systems, the methods, the computer-readable media, and the techniques disclosed herein are included in Table 5. As shown in Table 5, under high TST, the participant took about 1.23 more operations per minute than the participant did under low TST. However is this outcome model were identical to the true outcome mechanism, then getting more sleep (e.g., a high TST) may increase the participant's physical activity (e.g., verse a low TST) by an average of only 1 operation per minute. That is, 1.23 operations per minute would have been a naïve overestimate of the APTE of sleep duration on physical activity. Applying the more flexible RF technique yields qualitatively similar results in this empirical example.

TABLE 5

| | Estimate | Estimated Steps |
|---|---|---|
| Method | (95% CI) | per Minute (95% CI) |
| Raw comparison | 0.09 (0.02, 0.16) | 1.23 (1.05, 1.45) |
| MoTR-GLM | 0 (−0.07, 0.07) | 1 (0.851, 1.17) |
| PSTn (Stabilized)- | 0.15 | 1.41 |
| GLM | | |
| MoTR-RF | 0.03 (−0.05, 0.1) | 1.07 (0.891, 1.26) |
| PSTn-RF | 0.22 | 1.66 |

On the other hand, if the propensity model were identical to the true propensity mechanism, then getting more sleep would have increased the participant's physical activity by an average of 1.41 operations per minute. In this case, 1.23 operations per minute would have been a naïve underestimate of the APTE of sleep duration n physical activity. As with MoTR, the RF technique yields qualitatively similar results.

The results of at least this empirical study demonstrate the effectiveness and advantages of the systems, the methods, the computer-readable media, and the techniques disclosed herein. For example, presented herein is a model-twin randomization model applied to estimate the APTE using, e.g., wearable sensor data. Using this model, estimation of the daily APTE of sleep duration on operations per minute using 222 days of participant wearable data is presented as an empirical study.

In some cases, the contradiction in APTE estimates between MoTR and the propensity-score based technique “propensity score twin” results may highlight the importance of specifying the model as close to the true mechanism as possible. This may be done both empirically (e.g., in a data-driven way, such as using parameter tuning and model selection) and scientifically (e.g., based on related literature).

In some cases, an advanced doubly robust technique may be applied using the systems, the methods, the computer-readable media, and the techniques disclosed herein. Such advanced doubly robust technique may use targeted learning or targeted maximum likelihood estimation (TMLE) to estimate and infer the APTE. This TMLE technique may lay the groundwork for a sequentially adaptive design for learning optimal idiographic treatment rules over time. In some cases, the TMLE technique also optimizes the use of machine learning models by minimizing the bias incurred when fitting such models.

The systems, the methods, the computer-readable media, and the techniques disclosed herein provide the MoTR model as a way to surface plausible or suggested causal effects for possible further investigation or confirmation through an n-of-1 trial. The MoTR model can be used, in some cases, to develop a personalized idiographic intervention plan. For example, one might use the following data-driven procedure to identify combinations of models and plausible confounders that produce APTE estimates that meaningfully differ from naïve estimates calculated by comparing raw averages (as in Table 3). In some cases, one or more applications of the systems, the methods, the computer-readable media, and the techniques disclosed herein may include:
(1) the study participant, their health provider, or an analyst decide on plausible confounders;
(2) the analyst may fit and may select or cross-validate initial outcome or propensity models;
(3) the analyst may select the final model (e.g., test all selected models on a holdout set);
(4) the analyst runs MoTR or PSTn using these final models;
(5) the analyst reports the findings with the largest, most statistically discernible differences (e.g., with the highly statistically significant p-values) from the naïve estimates, where these findings may indicate situations in which confounding is strong enough to change effect estimates and thereby change how future within-individual interventions are designed; and
(6) the analyst discusses the plausibility of the selected models with the study participant and their health provider to determine an intervention plan.

In some cases, any number of elements of the applications described above may be added or removed. Further, the elements described above may occur in any order. Further, at least one of the elements described above may be repeated, e.g., iteratively.

In some cases, the APTE framework (e.g., MoTR and PSTn) may be applied to functional data analysis of intensive longitudinal data. For example, time point t(j) can include subpoints (e.g., t(jₖ)). This modularity allows for flexible temporal scaling of posited causal relationships. Accordingly, the APTE framework's utility can also be complemented and improved with formal causal diagrams such as directed acyclic graphs (DAGs) for conceptualizing formal structures. In some cases, a participant may use the systems, the methods, the computer-readable media, and the techniques disclosed herein to compare oneself to others; for example, one might ask, “how does my APTE relate to a corresponding group-level (nomothetic) ATE?” One common technique is to combine APTE estimates in the same way ATEs from multiple nomothetic studies may be aggregated in a meta-analysis (e.g., a series-of-n-of-1). Another technique may be to use a mixed-effects model to combine and compare participant−level APTEs to the overall ATE.

### Examples of Methods

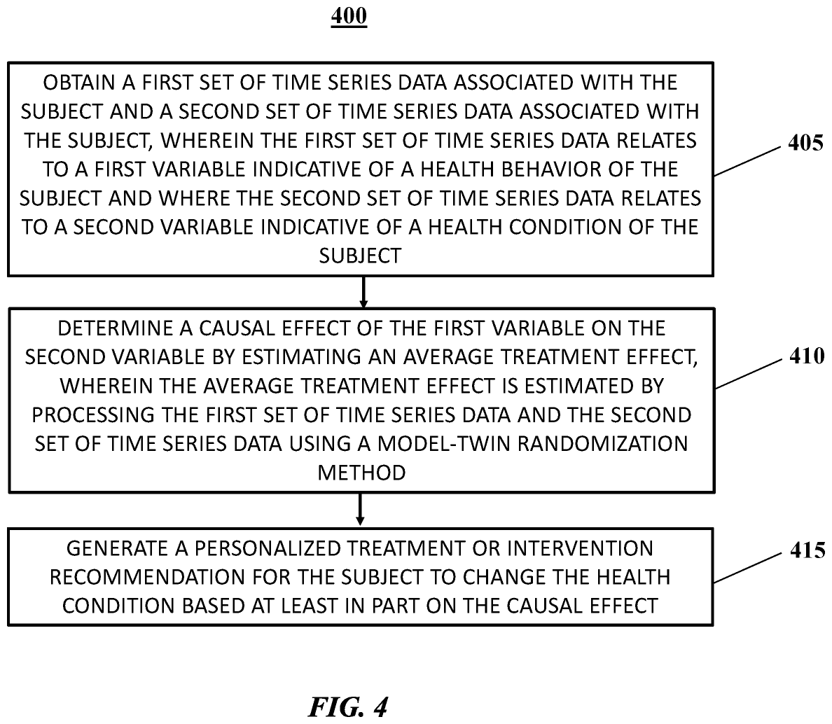

FIG. 4 shows an example of a flowchart illustrating a method 400 for generating a personalized recommended intervention for a subject based at least in part on causal inference. The method 400 may be implemented using one or more systems (e.g., hardware or software) described herein (e.g., the environment 100). The method 400 may implement one or more techniques or operations described herein. At a high level, the method 400 may include: obtaining a first set of time series data associated with the subject and a second set of time series data associated with the subject, where the first set of time series data relates to a first variable indicative of a health behavior of the subject and where the second set of time series data relates to a second variable indicative of a health condition of the subject (block 405); determining a causal effect of the first variable on the second variable by estimating an average treatment effect, where the average treatment effect is estimated by processing the first set of time series data and the second set of time series data using a model-twin randomization method (block 410); and generating a personalized treatment or intervention recommendation for the subject to change the health condition based at least in part on the causal effect determined in the block 410 (block 415).

In some cases, with the method 400, the model-twin randomization method comprises a sequential technique to implement g-formula for estimating the average treatment effect. In some cases, the sequential technique comprises a simulation-based technique or a Monte Carlo technique. In some cases, processing the first set of time series data and the second set of time series data using the model-twin randomization method comprises randomizing the first set of time series data for each time period or time point of the first set of time series data. In some cases, the method 400 further comprises running a model-twin through the randomized first set of time series data for a number of iterations until a convergence condition is satisfied. In some cases, the model-twin is an outcome model fitted to the first set of time series data and the second set of time series data. In some cases, the method 400 further comprises generating, by the model-twin, a predicted value of the second variable at each time point of the second set of time series data. In some cases, the method 400 further comprises adding random noise to each predicted value of the second variable. In some cases, the method 400 further comprises determining an average period treatment effect (APTE) from at least a subset of each of the predicted values for at least a subset of the set of time points. In some cases, the method 400 further comprises estimating a confidence interval for the APTE. In some cases, the method 400 further comprises calculating a cumulative average confidence interval, where the convergence condition relates to the cumulative average confidence interval. In some cases, the model-twin comprises a generalized linear model. In some cases, the linear model comprises a generalized linear model. In some cases, the model-twin comprises a non-parametric model. In some cases, the model-twin comprises a machine learning model. In some cases, the machine learning model comprises a random forest. In some cases, the first set of time series data is acquired from a wearable device worn by the subject. In some cases, the first set of time series data is indicative of sleep duration and where the second set of time series data is indicative of physical activity In some cases, the second set of time series data is indicative of speed of walking. In some cases, the second set of time series data is indicative of sleep duration and where the first set of time series data is indicative of physical activity. In some cases, one or both of the first set of time series data or the second set of time series data is collected daily. In some cases, one or both of the first set of time series data or the second set of time series data comprise variables that cause, moderate, or contextualize data comprised in one or both of the first set of time series data or the second set of time series data. In some cases, the personalized treatment or intervention recommendation comprises changing health behavior of the subject.

In some cases, any number of operations of the method 400 may be added or removed. Further, the operations of the method 400 may be performed in any order Further, at least one of the operations of the method 400 may be repeated, e.g., iteratively.

### Examples of Machine Learning Techniques

As disclosed throughout, in some cases, the systems, the methods, the computer-readable media, and the techniques disclosed herein may implement one or more machine learning techniques. In some cases, machine learning (ML) may generally involve identifying and recognizing patterns in existing data in order to facilitate making predictions for subsequent data. ML may include a ML model (which may include, for example, a ML algorithm). Machine learning, whether analytical or statistical in nature, may provide deductive or abductive inference based on real or simulated data. The ML model may be a trained model. ML techniques may comprise one or more supervised, semi-supervised, self-supervised, or unsupervised ML techniques. For example, an ML model may be a trained model that is trained through supervised learning (e.g., various parameters are determined as weights or scaling factors). ML may comprise one or more of regression analysis, regularization, classification, dimensionality reduction, ensemble learning, meta learning, association rule learning, cluster analysis, anomaly detection, deep learning, or ultra-deep learning. ML may comprise: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation (LASSO), least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t−distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short−term memory, deep belief networks, deep Boltzmann machines, deep convolutional neural networks, deep recurrent neural networks, large language models, vision transformers, or generative adversarial networks.

Training the ML model may include, in some cases, selecting one or more untrained data models to train using a training data set. The selected untrained data models may include any type of untrained ML models for supervised, semi-supervised, self-supervised, or unsupervised machine learning. The selected untrained data models may be specified based upon input (e.g., user input) specifying relevant parameters to use as predicted variables or other variables to use as potential explanatory variables. For example, the selected untrained data models may be specified to generate an output (e.g., a prediction) based upon the input. Conditions for training the ML model from the selected untrained data models may likewise be selected, such as limits on the ML model complexity or limits on the ML model refinement past a certain point. The ML model may be trained (e.g., via a computer system such as a server) using the training data set. In some cases, a first subset of the training data set may be selected to train the ML model. The selected untrained data models may then be trained on the first subset of training data set using appropriate ML techniques, based upon the type of ML model selected and any conditions specified for training the ML model. In some cases, due to the processing power requirements of training the ML model, the selected untrained data models may be trained using additional computing resources (e.g., cloud computing resources). Such training may continue, in some cases, until at least one aspect of the ML model is validated and meets selection criteria to be used as a predictive model.

In some cases, one or more aspects of the ML model may be validated using a second subset of the training data set (e.g., distinct from the first subset of the training data set) to determine accuracy and robustness of the ML model. Such validation may include applying the ML model to the second subset of the training data set to make predictions derived from the second subset of the training data. The ML model may then be evaluated to determine whether performance is sufficient based upon the derived predictions. The sufficiency criteria applied to the ML model may vary depending upon the size of the training data set available for training, the performance of previous iterations of trained models, or user-specified performance requirements. If the ML model does not achieve sufficient performance, additional training may be performed. Additional training may include refinement of the ML model or retraining on a different first subset of the training dataset, after which the new ML model may again be validated and assessed. When the ML model has achieved sufficient performance, in some cases, the ML may be stored for present or future use. The ML model may be stored as sets of parameter values or weights for analysis of further input (e.g., further relevant parameters to use as further predicted variables, further explanatory variables, further user interaction data, etc.), which may also include analysis logic or indications of model validity in some instances. In some cases, a plurality of ML models may be stored for generating predictions under different sets of input data conditions. In some embodiments, the ML model may be stored in a database (e.g., associated with a server).

### Examples of Decision Trees and Random Forests

As described above, the machine learning model may implement a decision tree. A decision tree may be a supervised ML algorithm that can be applied to both regression and classification problems. Decision trees may mimic the decision-making process of a human brain. For example, a decision tree may grow from a root (base condition), and when it meets a condition (internal node/feature), it may split into multiple branches. The end of the branch that does not split anymore may be an outcome (leaf). A decision tree can be generated using a training data set according to the following operations: (1) starting from a root node (the entire dataset), the algorithm may split the dataset in two branches using a decision rule or branching criterion, (2) each of these two branches may generate a new child node; (3) for each new child node, the branching process may be repeated until the dataset cannot be split any further; (4) each branching criterion may be chosen to maximize information gain (e.g., a quantification of how much a branching criterion reduces a quantification of how mixed the labels are in the children nodes). The labels may be the data or the classification that is predicted by the decision tree.

A random forest regression is an extension of the decision tree model that tends to yield more robust predictions by stretching the use of the training data partition. Whereas a decision tree may make a single pass through the data, a random forest regression may bootstrap 50% of the data (e.g., with replacement) and build many trees. Rather than using all explanatory variables as candidates for splitting, a random subset of candidate variables may be used for splitting, which may enable trees that have different data and different variables (hence the term random). The predictions from the trees, which may be collectively referred to as the “forest,” may then be averaged to produce a final prediction. Many trees (e.g., ten trees, fifty trees, one hundred trees, one thousand trees, etc.) may be included in a random forest model, with a number (e.g., 3, 6, 10, etc.) of terms sampled per split, a minimum of number (e.g., 1, 2, 4, 10, etc.) of splits per tree, and a minimum split size (e.g., 16, 32, 64, 128, 256, etc.). Random forests may be trained in a similar way as decision trees. Specifically, training a random forest may include the following operations: (1) randomly select k features from the total number of features; (2) create a decision tree from these k features using the same operations as for generating a decision tree; and (3) repeat the previous two operations until a target number of trees is created.

As disclosed, a random forest classifier, which may comprise a plurality of decision trees where the output prediction may be the mode of the predicted classifications of the individual trees, can be helpful in reducing overfitting to training data. In some cases, an ensemble of decision trees can be constructed using a random subset of features at each split or decision node. The Gini criterion may be employed, in some cases, to choose the best partition, where decision nodes having the lowest calculated Gini impurity index are selected. The Gini impurity can be used, in some cases, as a criterion to find informative features based on which the splits in each decision tree may be constructed.

In some cases, each decision tree of a random forest may comprise one or more decision nodes, where each decision node specifies a predicate condition. For example, decision node may predicate the condition that, for a given dataset, the outcome to an question is a specific outcome. At each decision node, a decision tree can be split based on whether the predicate condition attached to the decision node holds true, leading to various prediction nodes Each prediction node can comprise output values that represent “votes” for one or more of the classifications or conditions being evaluated by the assessment model. At prediction time, a “vote” can be taken over all of the decision trees, and the majority vote (or mode of the predicted classifications) can be output as the predicted classification.

In some cases, when the dataset being queried in the assessment model reaches a “leaf”, or a final prediction node with no further downstream splits, the output values of the leaf can be output as the votes for the particular decision tree. Since a random forest model comprises a plurality of decision trees, the final votes across all trees in the forest can be summed to yield the final votes and the corresponding classification of the subject. A large number of decision trees can help reduce overfitting of the assessment model to the training data, by reducing the variance of each individual decision tree. For example, an assessment model can comprise, for example, at least about 3 decision trees, at least about 5 decision trees, at least about 10 decision trees, at least about 20 decision trees, at least about 50 decision trees, at least about 100 decision trees, etc.

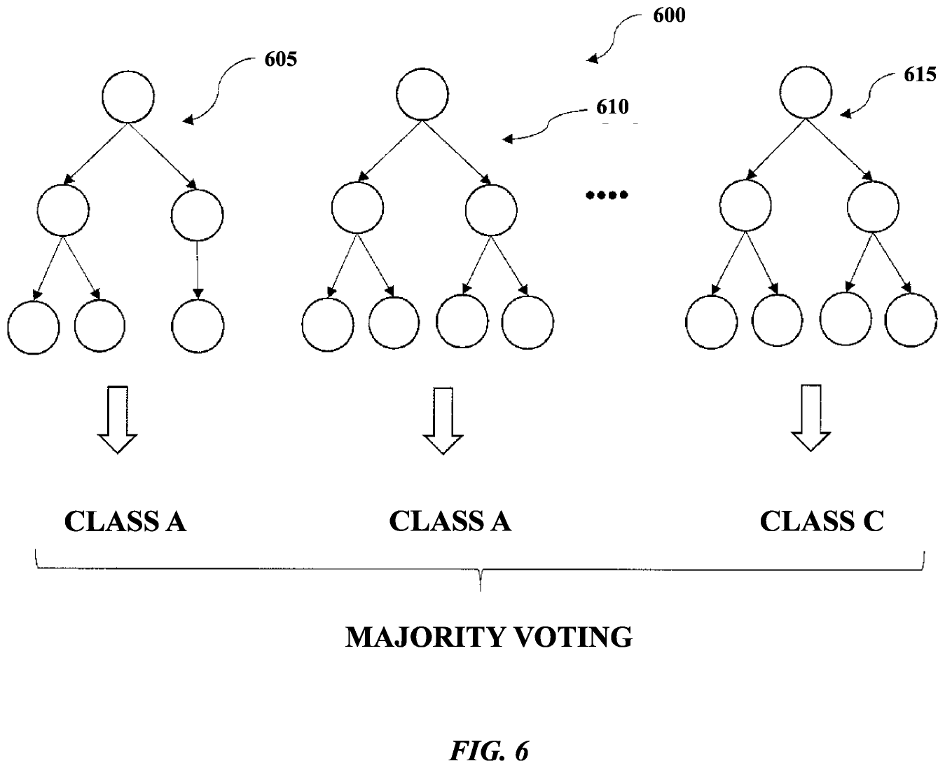

FIG. 6 illustrates a random forest 600. The random forest 600 (which may also be referred to as a random forest model) is an ensemble of decision trees 605, 610, and 615 with randomly selected features in each of the decision trees 605, 610, and 615 such that the random forest 600 can provide more stable and accurate outcomes. Outcomes may be determined by majority voting in the case of a classification problem. In the example of FIG. 6, the random forest 600, which has been trained previously by a training method, is used to decide between classifications A, B and C. For example, the random forest 600, with only the three decision trees shown in FIG. 6, would return the classification A by majority voting.

### Examples of Computing Systems

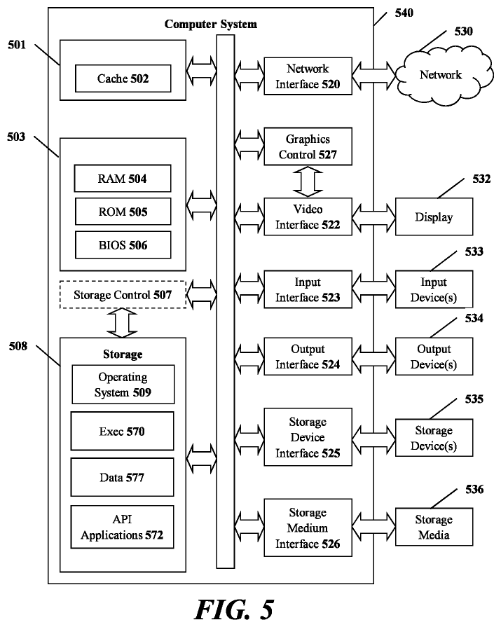

Referring to FIG. 5, a block diagram is shown depicting an example machine that includes a computer system 500 (e.g., a processing or computing system) within which a set of instructions can execute for causing a device to perform or execute any one or more of the aspects or methodologies for static code scheduling of the present disclosure. The components in FIG. 5 are examples and do not limit the scope of use or functionality of any hardware, software, embedded logic component, or a combination of two or more such components with particular implementations.

Computer system 500 may include one or more processors 501, a memory 503, and a storage 508 that communicate with each other, and with other components, via a bus 540. The bus 540 may also link a display 532, one or more input devices 533 (which may, for example, include a keypad, a keyboard, a mouse, a stylus, etc.), one or more output devices 534, one or more storage devices 535, and various tangible storage media 536. All of these elements may interface directly or via one or more interfaces or adaptors to the bus 540. For instance, the various tangible storage media 536 can interface with the bus 540 via storage medium interface 526. Computer system 500 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 500 includes one or more processor(s) 507 (e.g., central processing units (CPUs), general purpose graphics processing units (GPGPUs), or quantum processing units (QPUs)) that carry out functions. Processor(s) 501 optionally contains a cache memory unit 502 for temporary local storage of instructions, data, or computer addresses. Processor(s) 501 are configured to assist in execution of computer readable instructions. Computer system 500 may provide functionality for the components depicted in FIG. 5 as a result of the processor(s) 501 executing non-transitory, processor-executable instructions embodied in one or more tangible computer-readable storage media, such as memory 503, storage 508, storage devices 535, or storage medium 536. The computer-readable media may store software that implements particular operations, and processor(s) 501 may execute the software. Memory 503 may read the software from one or more other computer-readable media (such as mass storage device(s) 535, 536) or from one or more other sources through a suitable interface, such as network interface 520. The software may cause processor(s) 501 to carry out one or more processes or one or more operations of one or more processes described or illustrated herein. Carrying out such processes or operations may include defining data structures stored in memory 503 and modifying the data structures as directed by the software.

The memory 503 may include various components (e.g., machine readable media) including, but not limited to, a random access memory component (e.g., RAM 504) (e.g., static RAM (SRAM), dynamic RAM (DRAM), ferroelectric random access memory (FRAM), phase-change random access memory (PRAM), etc.), a read-only memory component (e.g., ROM 505), and any combinations thereof. ROM 505 may act to communicate data and instructions unidirectionally to processor(s) 501, and RAM 504 may act to communicate data and instructions bidirectionally with processor(s) 501. ROM 505 and RAM 504 may include any suitable tangible computer-readable media described below. In one example, a basic input/output system 506 (BIOS), including basic routines that help to transfer information between elements within computer system 500, such as during start−up, may be stored in the memory 503.

Fixed storage 508 is connected bidirectionally to processor(s) 501, optionally through storage control unit 507. Fixed storage 508 provides additional data storage capacity and may also include any suitable tangible computer-readable media described herein. Storage 508 may be used to store operating system 509, executable(s) 510, data 511, applications 512 (application programs), and the like. Storage 508 can also include an optical disk drive, a solid-state memory device (e.g., flash-based systems), or a combination of any of the above. Information in storage 508 may, in appropriate cases, be incorporated as virtual memory in memory 503.

In one example, storage device(s) 535 may be removably interfaced with computer system 500 (e.g., via an external port connector (not shown)) via a storage device interface 525. Particularly, storage device(s) 535 and an associated machine-readable medium may provide non-volatile or volatile storage of machine-readable instructions, data structures, program modules, or other data for the computer system 500. In one example, software may reside, completely or partially, within a machine-readable medium on storage device(s) 535. In another example, software may reside, completely or partially, within processor(s) 501.

Bus 540 connects a wide variety of subsystems. Herein, reference to a bus may encompass one or more digital signal lines serving a common function, where appropriate. Bus 540 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 500 may also include an input device 533. In one example, a user of computer system 500 may enter commands or other information into computer system 500 via input device(s) 533. Examples of an input device(s) 533 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some cases, the input device is a Kinect, Leap Motion, or the like. Input device(s) 533 may be interfaced to bus 540 via any of a variety of input interfaces 523 (e.g., input interface 523) including, but not limited to, serial, parallel, game port, USB, FIREWIRE, THUNDERBOLT, or any combination of the above.

In some cases, when computer system 500 is connected to network 530, computer system 500 may communicate with other devices, specifically mobile devices and enterprise systems, distributed computing systems, cloud storage systems, cloud computing systems, and the like, connected to network 530. Communications to and from computer system 500 may be sent through network interface 520. For example, network interface 520 may receive incoming communications (such as requests or responses from other devices) in the form of one or more packets (such as Internet Protocol (IP) packets) from network 530, and computer system 500 may store the incoming communications in memory 503 for processing. Computer system 500 may similarly store outgoing communications (such as requests or responses to other devices) in the form of one or more packets in memory 503 and communicated to network 530 from network interface 520. Processor(s) 501 may access these communication packets stored in memory 503 for processing.

Examples of the network interface 520 include, but are not limited to, a network interface card, a modem, and any combination thereof. Examples of a network 530 or network segment 530 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 530, may employ a wired or a wireless mode of communication. In general, any network topology may be used.

Information and data can be displayed through a display 532. Examples of a display 532 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 532 can interface to the processor(s) 501, memory 503, and fixed storage 508, as well as other devices, such as input device(s) 533, via the bus 540. The display 532 is linked to the bus 540 via a video interface 522, and transport of data between the display 532 and the bus 540 can be controlled via the graphics control 521. In some cases, the display is a video projector. In some cases, the display is a head-mounted display (HMD) such as a VR headset. In further cases, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further cases, the display is a combination of devices such as those disclosed herein.

In addition to a display 532, computer system 500 may include one or more other peripheral output devices 534 including, but not limited to, an audio speaker, a printer, a storage device, and any combinations thereof. Such peripheral output devices may be connected to the bus 540 via an output interface 524. Examples of an output interface 524 include, but are not limited to, a serial port, a parallel connection, a USB port, a FIREWIRE port, a THUNDERBOLT port, and any combinations thereof.

In addition or as an alternative, computer system 500 may provide functionality as a result of logic hardwired or otherwise embodied in a circuit, which may operate in place of or together with software to execute one or more processes or one or more operations of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Various illustrative logical blocks, modules, circuits, and algorithm operations described in connection with the examples disclosed herein may be implemented as electronic hardware, computer software, or combinations of both. To clearly illustrate this interchangeability of hardware and software, various illustrative components, blocks, modules, circuits, and operations have been described above generally in terms of their functionality.

The various illustrative logical blocks, modules, and circuits described in connection with the examples disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The operations of a method, a technique, or an algorithm described in connection with the examples disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An example storage medium may be coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set−top computers, media streaming devices, handheld computers, Internet appliances, mobile smartphones, tablet computers, personal digital assistants, video game consoles, and vehicles. Select televisions, video players, and digital music players with optional computer network connectivity may be suitable for use in the system described herein. Suitable tablet computers, in various cases, include those with booklet, slate, and convertible configurations.

In some cases, the computing device includes an operating system configured to perform executable instructions. The operating system is, for example, software, including programs and data, which manages the device's hardware and provides services for execution of applications. Suitable server operating systems may include, by way of non-limiting examples, FreeBSD, OpenBSD, NetBSD®, Linux, Apple® Mac OS X Server®, Oracle® Solaris®, Windows Server®, and Novell® NetWare®. Suitable personal computer operating systems may include, by way of non-limiting examples, Microsoft® Windows®, Apple® Mac OS X®, UNIX®, and UNIX-like operating systems such as GNU/Linux®. In some cases, the operating system is provided by cloud computing. Suitable mobile smartphone operating systems may include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some cases, the systems, the methods, the computer-readable media, and the techniques disclosed herein include one or more non-transitory computer readable storage media encoded with a program including instructions executable by the operating system of an optionally networked computing device. In further cases, a computer readable storage medium is a tangible component of a computing device. In still further cases, a computer readable storage medium is optionally removable from a computing device. In some cases, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

In some cases, the systems, the methods, the computer-readable media, and the techniques disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. A computer program may be written in various versions of various languages.

The functionality of the computer readable instructions may be combined or distributed in various ways across various environments. In some cases, a computer program comprises one sequence of instructions. In some cases, a computer program comprises a plurality of sequences of instructions. In some cases, a computer program is provided from one location. In some cases, a computer program is provided from a plurality of locations. In some cases, a computer program includes one or more software modules. In some cases, a computer program includes, in part or in whole, one or more web applications, one or more mobile applications, one or more standalone applications, one or more web browser plug-ins, extensions, add-ins, or add-ons, or combinations thereof.

In some cases, a computer program includes a web application. A web application, in various cases, may utilize one or more software frameworks and one or more database systems. In some cases, a web application is created upon a software framework such as Microsoft® NET or Ruby on Rails (RoR). In some cases, a web application utilizes one or more database systems including, by way of non-limiting examples, relational, non-relational, object oriented, associative, XML, and document oriented database systems. In further cases, suitable relational database systems include, by way of non-limiting examples, Microsoft® SQL Server, mySQL™, and Oracle®. A web application, in some cases, may be written in one or more versions of one or more languages. A web application may be written in one or more markup languages, presentation definition languages, client−side scripting languages, server-side coding languages, database query languages, or combinations thereof. In some cases, a web application is written to some extent in a markup language such as Hypertext Markup Language (HTML), Extensible Hypertext Markup Language (XHTML), or extensible Markup Language (XML). In some cases, a web application is written to some extent in a presentation definition language such as Cascading Style Sheets (CSS). In some cases, a web application is written to some extent in a client−side scripting language such as Asynchronous JavaScript and XML (AJAX), Flash® ActionScript, JavaScript, or Silverlight®. In some cases, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some cases, a web application is written to some extent in a database query language such as Structured Query Language (SQL). In some cases, a web application integrates enterprise server products such as IBM® Lotus Domino®. In some cases, a web application includes a media player element. In some cases, a media player element utilizes one or more of many suitable multimedia technologies including, by way of non-limiting examples, Adobe® Flash®. HTML 5, Apple® QuickTime®, Microsoft® Silverlight®, Java™, and Unity®.

In some cases, a computer program includes a mobile application provided to a mobile computing device. In some cases, the mobile application is provided to a mobile computing device at the time it is manufactured. In other cases, the mobile application is provided to a mobile computing device via the computer network described herein.

In view of the disclosure provided herein, a mobile application may be created using hardware, languages, and development environments known to the art. In some cases, mobile applications are written in several languages. Suitable programming languages may include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and PhoneGap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Several commercial forums may be available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, and Samsung Apps.

In some cases, a computer program includes a standalone application, which is a program that is run as an independent computer process, not an add-on to an existing process, e.g., not a plug-in. Standalone applications may be compiled. A compiler may be a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some cases, a computer program includes one or more executable complied applications.

In some cases, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Web browser plug-ins may include Adobe® Flash® Player, Microsoft® Silverlight®, and Apple® QuickTime®. In some cases, the toolbar comprises one or more web browser extensions, add-ins, or add-ons. In some cases, the toolbar comprises one or more explorer bars, tool bands, or desk bands.

Several plug-in frameworks may be available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari® Opera Software® Opera®, and KDE Konqueror. In some cases, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, MozillavFirefox® for mobile, Microsoft® Internet Explorer® Mobile, Amazon® Kindle® Basic Web, Nokia® Browser, Opera Software® Opera® Mobile, and Sony® PSP™ browser.

In some cases, the systems, the methods, the computer-readable media, and the techniques disclosed herein include software, server, or database modules, or use of the same. Software modules may be created by techniques using machines, software, and languages. The software modules disclosed herein are implemented in a multitude of ways. In some cases, a software module comprises a file, a section of code, a programming object, a programming structure, a distributed computing resource, a cloud computing resource, or combinations thereof. In some cases, a software module comprises a plurality of files, a plurality of sections of code, a plurality of programming objects, a plurality of programming structures, a plurality of distributed computing resources, a plurality of cloud computing resources, or combinations thereof. In some cases, the one or more software modules comprise, by way of non-limiting examples, a web application, a mobile application, a standalone application, and a distributed or cloud computing application. In some cases, software modules are in one computer program or application. In some cases, software modules are in more than one computer program or application. In some cases, software modules are hosted on one machine. In some cases, software modules are hosted on more than one machine. In some cases, software modules are hosted on a distributed computing platform such as a cloud computing platform. In some cases, software modules are hosted on one or more machines in one location. In some cases, software modules are hosted on one or more machines in more than one location.

In some cases, the systems, the methods, the computer-readable media, and the techniques disclosed herein include one or more databases, or use of the same. In some cases, various databases may be suitable for storage and retrieval of one or more of (i) wearable data, (ii) responses to health queries, (iii) geographic data, etc., one or more of which may be historical, present, or future data or information. In some cases, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some cases, a database is Internet−based. In further cases, a database is web-based. In still further cases, a database is cloud computing-based. In a particular case, a database is a distributed database. In other cases, a database is based on one or more local computer storage devices.

### Additional Considerations

While various embodiments of the invention have been shown and described herein, it will be obvious to those skilled in the art that such embodiments are provided by way of example. Numerous variations, changes, and substitutions may occur to those skilled in the art without departing from the invention. It should be understood that various alternatives to the embodiments of the invention described herein may be employed.

It should be noted that various illustrative or suggested ranges set forth herein are specific to their example embodiments and are not intended to limit the scope or range of disclosed technologies, but, again, merely provide example ranges for frequency, amplitudes, etc. associated with their respective embodiments or use cases. Certain inventive embodiments herein contemplate numerical ranges. When ranges are present, the ranges include the range endpoints. Additionally, every sub range and value within the range is present as if explicitly written out.

While preferred embodiments of the present invention have been shown and described herein, it will be obvious to those skilled in the art that such embodiments are provided by way of example. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method for generating a personalized recommended intervention for a subject based at least in part on causal inference, comprising:
(a) obtaining a first set of time series data associated with the subject and a second set of time series data associated with the subject, wherein the first set of time series data relates to a first variable indicative of a health behavior of the subject and wherein the second set of time series data relates to a second variable indicative of a health condition of the subject;
(b) determining a causal effect of the first variable on the second variable by estimating an average treatment effect, wherein the average treatment effect is estimated by processing the first set of time series data and the second set of time series data using a model-twin randomization method: and
(c) generating a personalized treatment or intervention recommendation for the subject to change the health condition based at least in part on the causal effect determined in (b).

2. The method of claim 1, wherein the model-twin randomization method comprises a sequential technique to implement g-formula for estimating the average treatment effect.

3. The method of claim 2, wherein implementing the g-formula comprises implementing extensions of the g-formula, wherein the extensions comprise one or both of targeted learning or targeted maximum likelihood estimation

4. The method of claim 2, wherein the sequential technique comprises a simulation-based technique or a Monte Carlo technique.

5. The method of claim 1, wherein processing the first set of time series data and the second set of time series data using the model-twin randomization method comprises randomizing the first set of time series data for each time period or time point of the first set of time series data.

6. The method of claim 5, further comprising running a model-twin through the randomized first set of time series data for a number of iterations until a convergence condition is satisfied.

7. The method of claim 6, wherein the model-twin is an outcome model fitted to the first set of time series data and the second set of time series data.

8. The method of claim 7, further comprising generating, by the model-twin, a predicted value of the second variable at each time point of the second set of time series data.

9. The method of claim 8, further comprising:
adding random noise to each predicted value of the second variable.

10. The method of claim 9, further comprising:
determining an average period treatment effect (APTE) from at least a subset of each of the predicted values for at least a subset of the set of time points.

11. The method of claim 10, further comprising:
estimating a confidence interval for the APTE.

12. The method of claim 11, further comprising:
calculating a cumulative average confidence interval, wherein the convergence condition relates to the cumulative average confidence interval.

13. The method of claim 6, wherein the model-twin comprises a generalized linear model.

14. The method of claim 13, wherein the linear model comprises a generalized linear model.

15. The method of claim 6, wherein the model-twin comprises a non-parametric model.

16. The method of claim 6, wherein the model-twin comprises a machine learning model.

17. The method of claim 15, wherein the machine learning model comprises a random forest.

18. The method of claim 1, wherein the first set of time series data is acquired from one or more data-collection instruments that comprise at least one wearable device worn by the subject.

19. The method of claim 18, wherein the first set of time series data is indicative of sleep duration and wherein the second set of time series data is indicative of physical activity.

20. The method of claim 18, wherein the second set of time series data is indicative of speed of walking.

21. The method of claim 18, wherein the second set of time series data is indicative of sleep duration and wherein the first set of time series data is indicative of physical activity.

22. The method of claim 18, wherein one or both of the first set of time series data or the second set of time series data is collected daily.

23. The method of claim 1, wherein one or both of the first set of time series data or the second set of time series data comprise variables that cause, moderate, or contextualize data comprised in one or both of the first set of time series data or the second set of time series data.

24. The method of claim 1, wherein the personalized treatment or intervention recommendation comprises changing health behavior of the subject.

25. The method of claim 24, wherein changing the health behavior or the subject comprises estimating a plausible or suggested average treatment effect of the health behavior of the subject on the health condition of the subject.

26. One or more non-transitory computer-readable media comprising computer-executable instructions that, when executed by at least one processor, cause the at least one processor to:
(a) obtain a first set of time series data associated with the subject and a second set of time series data associated with the subject, wherein the first set of time series data relates to a first variable indicative of a health behavior of the subject and wherein the second set of time series data relates to a second variable indicative of a health condition of the subject;
(b) determine a causal effect of the first variable on the second variable by estimating an average treatment effect, wherein the average treatment effect is estimated by processing the first set of time series data and the second set of time series data using a model-twin randomization method; and
(c) generate a personalized treatment or intervention recommendation for the subject to change the health condition based at least in part on the causal effect determined in (b).

27. A computer system for generating a personalized recommended intervention for a subject based at least in part on causal inference, comprising:
one or more processors; and
one or more memories storing computer-executable instructions that, when executed, cause the one or more processors to:
(a) obtain a first set of time series data associated with the subject and a second set of time series data associated with the subject, wherein the first set of time series data relates to a first variable indicative of a health behavior of the subject and wherein the second set of time series data relates to a second variable indicative of a health condition of the subject;
(b) determine a causal effect of the first variable on the second variable by estimating an average treatment effect, wherein the average treatment effect is estimated by processing the first set of time series data and the second set of time series data using a model-twin randomization method; and
(c) generate a personalized treatment or intervention recommendation for the subject to change the health condition based at least in part on the causal effect determined in (b).
